(19) United States
(12) Patent Application Publication
(10) Pub. No.: US 2005/0100899 A1
(43) Pub. Date: May 12, 2005

(54) Screening assays

(75) Inventors: Stuart Knutton, Birmingham (GB); Gad Meir Frankel, London (GB)

Correspondence Address: NIKOLAI & MERSEREAU, P.A., 900 SECOND AVENUE SOUTH, SUITE 820, MINNEAPOLIS, MN, 55402, US

(73) Assignee: Imperial College Innovations Limited, London (GB)

(21) Appl. No.: 10/429,892

(22) Filed: Apr. 25, 2003

Related U.S. Application Data
(63) Continuation of application No. PCT/GB01/04684, filed on Oct. 22, 2001.

(51) Int. Cl. C12Q 1/68

(52) U.S. Cl. 435/6

(57) ABSTRACT

A method of analysing gene expression occurring in a microorganism before, during or after contact with or adhesion of the microorganism to a lipid bilayer, comprising the step of exposing the microorganism to a lipid bilayer, wherein the lipid bilayer is substantially not associated with protein or RNA synthetic machinery.

The lipid bilayer may be a red blood cell membrane, for example in the form of intact red blood cells. The red blood cells may be immobilised as a monolayer. The microorganism may be an enteropathogenic or enterohaemorrhagic E. coli. A DNA or protein microarray may be used in analysing gene expression.

The present invention relates to screening assays, in particular screening assays related to identification of antiinfective agents and targets therefor, in particular antibacterial agents and targets therefor.

Most putative pathogen, for example bacterial, virulence factors are either located at the pathogen surface or are secreted from the pathogen, for example bacterium, sometimes directly into host cells. For some bacteria, translocation of virulence proteins into host cells is dependent upon possession of a specialised secretion system, for example a type III secretion system, which has been found in many Gram-negative bacteria (Hueck, 1998). Heliobacter spp, for example, have a type IV secretion system. In contrast, Campylobacter spp are not thought to possess a secretion system. Enteropathogenic E. coli (EPEC) is a bacterial pathogen that employs a multi-stage infection strategy involving type III secretion and protein translocation into host cells (Frankel et al., 1998). An established etiological agent of human infantile diarrhoea, EPEC subvert intestinal epithelial cell function to produce distinctive “attaching & effacing” (A/E) lesions, characterised by localised destruction (effacement) of brush border microvilli, intimate bacterial attachment to the host cell membrane and formation of an actin-rich cytoskeletal structure beneath intimately attached bacteria (Frankel, et al., 1998); similar lesions are produced in a variety of cultured epithelial cell lines (Knutton et al., 1987). All the genes required for A/E lesion formation are encoded by the LEE (locus of enterocyte effacement) pathogenicity island (McDaniel & Kaper, 1997), which encodes the type III secretion apparatus (Jarvis et al., 1995), the intimate EPEC adhesin, intimin (Jerse et al 1990), secreted proteins EspA, EspB, EspD (Frankel et al 1998) and a translocated intimin receptor, Tir (Kenny et al 1997).

Type III secretion systems demonstrate a broad functional conservation across bacterial species and many of the components show similarity to proteins involved in flagellar biosynthesis (Hueck, 1998). Type III secretion systems (secretons) have recently been visualised by electron microscopy (Blocker et al 1999; Kubori et al 1998; Tamano et al 2000); they comprise a macromolecular complex spanning both bacterial membranes with an external needle structure (Blocker et al 1999; Tamano et al 2000). In addition to a type III secreton, injection of proteins into host cells is thought to require insertion of pore-forming proteins into the host cell membrane (Hueck, 1998). In Yersinia these are the type III secreted YopB/D proteins (Neyt & Comelis, 1999) and in Shigella the IpaB/C proteins (Blocker et al 1999). Injection of proteins into host cells by Shigella and Yersinia spp. has been correlated with their ability to cause contact dependent haemolysis of red blood cells (RBC) in vitro in which bacteria are brought into close contact with the RBC membrane by centrifugation (Clerc et al 1986; Hakansson et al 1996).

The EPEC type III secreton has yet to be visualised although, based on homology with other type III secretion system proteins, it is likely to have a structure similar to that described for Shigella ie a macromolecular complex spanning both bacterial membranes (Blocker et al., 1999). Four proteins essential for A/E lesion formation are known to be secreted by the EPEC type III secreton; EspA, EspB, EspD, and Tir. Tir is translocated and inserted into the host cell membrane where it functions as a receptor for the intimate bacterial adhesin, intimin (Deibel et al 1998; Kenny et al 1997); EspA, EspB, and EspD are thought to be components of the translocation apparatus. Secreted EspB (Wolff et al 1998) and EspD (Wachter et al 1999) have been shown to be delivered to the host cell membrane and, based on homology with Ipa B/C and Yop B/D, proposed to form a pore complex in the host membrane (Frankel et al 1998). EspA is the major component of a large filamentous structure which is proposed to provide a conduit between the type III translocon and a host cell membrane pore (Knutton et al 1998).

EPEC were recently shown to exhibit a contact-dependent haemolytic activity (Warawa et al 1999).

We show that the interaction between an attaching microorganism, for example a pathogenic organism, and a lipid bilayer which is substantially not associated with protein or RNA synthetic machinery, may be used as a model in identifying changes in levels of cell components, particularly changes in protein or RNA expression, in the attaching organism associated with early interaction between the organism and a host cell. The identified components, for example proteins, may be targets for vaccines and/or compounds that may modulate, preferably inhibit, the interaction between the attaching organism and a host cell. The identified components may be useful in relation to diagnosis, for example in identification of the microorganism(s) involved in an infection.

The model may be particularly useful in relation to enteropathogenic E. coli (EPEC) or Enterohaemorrhagic E. coli (EHEC) or other organisms in which close contact between the attaching organism and the lipid bilayer, for example induced by co-centrifugation, is not necessary for an interaction to occur. The interaction may therefore advantageously be investigated using immobilised lipid bilayers. This may facilitate separation of organisms adhering to the lipid bilayer from organisms that are not so adhering.

A first aspect of the invention provides a method of analysing gene expression occurring in a microorganism before, during or after contact with or adhesion of the microorganism to a lipid bilayer, comprising the step of exposing the microorganism to a lipid bilayer, wherein the lipid bilayer is substantially not associated with protein or RNA synthetic machinery.

The contact with or adhesion of the microorganism to the lipid bilayer may take the form of the microorganism invading or passing through the lipid bilayer. Thus, the term contact/adhesion also encompasses invasion or passing through the lipid bilayer; and the invention provides a method of analysing gene expression occurring in a microorganism before, during or after invasion of the microorganism through a lipid bilayer, comprising the step of exposing the microorganism to a lipid bilayer, wherein the lipid bilayer is substantially not associated with protein or RNA synthetic machinery. Invasive microorganisms include plasmodium and Bartonella.

The lipid bilayer may comprise non-lipid components, for example polypeptides, including glycoproteins. It is preferred that the lipid bilayer is comprised in a red blood cell (erythrocyte) ie is a red blood cell plasma membrane, or is a membrane derived from a red blood cell. As indicated in Example 1, the initial contact between microorganisms, for example enteropathogenic or enterohaemorrhagic E. coli (EPEC or EHEC) and red blood cells is considered to mimic the initial contact between such microorganisms and host cells to which the microorganisms may normally bind, for example cells of the gut.

It will be appreciated that red blood cells are substantially devoid of protein synthetic machinery or RNA synthetic machinery; thus a red blood cell membrane is an example of a lipid bilayer that is substantially not associated with protein or RNA synthetic machinery. The terms protein synthetic machinery or RNA synthetic machinery will be well known to those skilled in the art. A cell which does not comprise a nucleus may be considered to be substantially devoid of RNA synthetic machinery (ie RNA polymerases and associated polypeptides/nucleic acids, as known to those skilled in the art). A cell from which the cytoplasm has been removed may be considered to be substantially devoid of protein synthetic machinery (ie ribosomes and associated polypeptides/nucleic acids, as known to those skilled in the art).

The lipid bilayer may be a purified cell membrane, ie a cell membrane which is substantially no longer associated with protein or RNA synthetic machinery. However, this is not preferred, because it may be more convenient to use red blood cells or membranes derived therefrom (or artificial lipid bilayers, as discussed below) than to perform the steps necessary to sufficiently dissociate the membrane from other cellular components.

The membrane derived from a red blood cell may be a red blood cell ghost, as well known to those skilled in the art. Ghosts may be prepared by lysis of red blood cells, for example by osmotic shock, for example as described in Example 1. Methods of preparing red blood cell membranes are well known to those skilled in the art, and are described in Example 1 and references therein. It is preferred that the red blood cell is a human red blood cell, particularly if it is desired to investigate the infection of humans by the microorganism, but this is not essential; any species may be chosen, so long as the microorganism is capable of binding to the chosen species of red blood cell.

Alternatively, the lipid bilayer may be an artificial lipid bilayer. Methods of preparing lipid bilayers are well known to those skilled in the art. It is preferred that the composition of the lipid bilayer resembles that of a eukaryotic cell, preferably that of a red blood cell. For example, the lipid bilayer may comprise the phospholipid dimyristoylphosphatidylcholine. The following references may describe suitable methods and compositions for preparing such lipid bilayers: Bartosz (1981) Biochim Biophys Acta 644(1), 69-73; Ingraham et al (1981) J Cell Biol 89(3), 510-516; Che et al (1997) Biochemistry 36(31), 9588-9595; Sakaki et al (1982) Biochemistry 21(10), 2366-2372; Rodgers & Glaser (1993) Biochemistry 32(47), 12591-12598.

It is strongly preferred that the method comprises the step of separating any microorganisms bound to the lipid bilayer from any microorganisms not so bound, following exposure of the microorganisms to the lipid bilayer, or any other means of analysing bound and unbound microorganisms separately (for example, single-cell analysis, as known to those skilled in the art). The adherent microorganisms may form a synchronised population, the members of which are at the same “developmental stage”, for example at the same stage of Type III secretion system activity. This may aid identification of differences in gene expression. The separation may be achieved by a variety of methods, as known to the skilled person, depending upon the microorganism in question and the form of the lipid bilayer. Conveniently, it may be achieved by immobilisation of the lipid bilayer (and hence of any microorganisms attached to the lipid bilayer). For example, if red blood cells or ghosts are used, then they may be immobilised, preferably as a monolayer, for example on a positively charged surface, such as a polylysine coated surface, for example as described in Example 1. Knutton & Baichi (1980) J Cell Sci 42, 153-167 describes suitable methods for the preparation of monolayers of red blood cells, unsealed (leaky) ghosts and resealed ghosts.

Magnetic separation methods may be used, for example immuno-magnetic separating methods, as well known to those skilled in the art. Thus, the lipid bilayer, for example red blood cell, may be attached to a magnetic bead, for example via an antibody/antigen interaction, with the result that bound microorganisms are also attached to the magnetic bead and can therefore be separated from unbound microorganisms in the surrounding fluid. Alternatively, microorganisms unbound by the lipid bilayer may be attached to the magnetic bead and thereby separated from microorganisms bound to the lipid bilayer.

The adherent microorganisms may then be separated from the lipid bilayer, for example by differential washing, but this may not be essential, depending upon the techniques to be used in analysis of gene expression. For example, if the analysis involves investigation of levels and/or types of proteins present and the lipid bilayer is in the form of red blood cells (which contain proteins), then it may be desirable to separate the adherent microorganisms from the red blood cells prior to analysis of the protein content of the micororganisms.

It may also be desirable to determine whether any microorganism component, for example protein, has been transferred to the lipid bilayer. Suitable methods are described, for example, in Example 1. For example red blood cells may be lysed and membranes separated, for example using a sucrose gradient (including step gradient) technique. Microorganism (for example, bacterial) protein may be identified by, for example, N-terminal sequencing.

The method may preferably comprise the step of comparing gene expression in the said microorganisms bound to the lipid bilayer with gene expression in the said microorganisms not so bound.

It is strongly preferred that gene expression is analysed by investigating the identities (ie hybridisation properties and/or sequences) and levels of nucleic acid, particularly mRNA in the microorganism, as well known to those skilled in the art. mRNA may conveniently be analysed in the form of cDNA, as well known to those skilled in the art. It is preferred that a method capable of automated and/or high throughput analysis is used. For example, the analysis may be performed using an array of immobilised nucleic acids (preferably DNA), for example a nucleic acid microarray. The immobilised nucleic acids may be gene-specific PCR products, for example as described in Example 2. Nucleic acid microarrays are well known to those skilled in the art, and allow simultaneous analysis of large numbers of nucleic acid sequences. Taton et al (2000) Science 289, 1757-1760, for example, describes sensitive labelling techniques that may be used with DNA arrays. Example 2 indicates other suitable methods that may be used. The nucleic acids may be immobilised on any suitable substrate, for example nitrocellulose or nylon membranes, or, more preferably, glass. Examples of references relating to DNA microarrays and methods of using same to analyse gene expression, for example detect differences in gene expression in two or more samples are as follows: EP 0 804 731; W092/10588; Gress et al (1992) Mammalian Genome 3, 609-619; U.S. Pat. No. 5,252,743 (all incorporated herein by reference).

When EPEC or EHEC strains are to be analysed, the DNA microarray may comprise nucleic, acids corresponding to the entire E. coli genome (ie corresponding to all genes identified in the E. coli genome), with the addition of EPEC and EHEC virulence genes, as indicated in Example 2.

Alternatively, gene expression may be analysed by investigating the types and levels of other components, for example polypeptides (including, for example, glycoproteins and lipoproteins) in the microorganism. It is preferred that a method capable of automated and/or high throughput analysis is used. For example, the analysis may be performed using an array of proteins, for example immobilised antibody fragments (for example scFvs), for example a protein or antibody fragment microarray. Protein microarrays and their use in investigating protein expression are discussed in the following references: de Wildt et al (2000) Antibody arrays for high-throughput screening of antibody-antigen interactions Nature Biotech 18, 989-994; Irving & Hudson (2000) Nature Biotech 18, 932-933; MacBeath & Schreiber (2000) Science 289, 1760-1763; Service (2000) Science 289, 1673. Protein arrays, particularly antibody arrays, may allow simultaneous analysis of multiple or large numbers of antigenic molecules, for example polypeptides. Other techniques well known to those skilled in the art may also be used, for example techniques using 2D gel electrophoresis.

As noted above, the microorganism may be a bacterium, for example a bacterium that is pathogenic or potentially pathogenic to animals, including humans. However, the microorganism may alternatively be a fungus, for example a fungus that is pathogenic or potentially pathogenic to animals, including humans. For example, the fungus may be any one of Aspergillus spp., Cryptococcus neoformans and Histoplasma capsulatum. The microorganism may be any other class of microorganism that adheres to animal cells, for example protozoa or trypanosomes or Plasmodium spp.

It is preferred that the bacterium is an E. coli bacterium, still more preferably an enterohaemorrhagic E. coli (EHEC) or an enteropathogenic E. coli (EPEC), yet more preferably EPEC strain E2348/69 (0127:H6) or EHEC strain 85-170 (O157:H7). These terms are well known to those skilled in the art (see, for example, Levine (1987) J Infect Dis 155, 377-389; WHO (1987), WHO Geneva, World Heath Organization 27; Orskov et al (1990) J Infect Dis 162, 76-81; Whittam et al (1993) Infect Immun 61, 1619-1629; Riley et al (1983) N Engl J Med 308, 681-685; Sherman et al (1988) Infect Immun 56, 756-761. The bacterium may be Citrobacter rodentium, which infects mice and may be closely related to EHEC and/or EPEC bacteria (Barthold et al (1976) Lab Anim Sci 26, 889-894; Schauer & Falklow (1993) Infect Immun 61, 4654-4661) or it may be a rabbit-specific EPEC strain, for example rabbit diarrhoeagenic E. coli (REPEC or RDEC-1; Cantey & Blake (1977) J Infect Dis 135, 454-462). Bacterial strains are available from, for example, the American Type Culture Collection (ATCC) of Rockville, Md., USA.

The bacterium may be a Helicobacter spp, for example H. pylon bacterium, as well known to those skilled in the art. Such bacteria may be involved in the development of gastrointestinal conditions, for example ulcers.

Alternatively, the bacterium may be, for example, any one of Bordetella pertussis, Campylobacter jejuni, Clostridium botulinum, Haemophilus ducreyi, Haemophilus influenzae, Klebsiella pneumoniae, Legionella pneumophila, Listeria spp., Neisseria gonorrhoeae, Neisseria meningitidis, Pseudomonas spp., Salmonella spp., Shigella spp., Staphylococcus aureus, Streptococcus pyogenes, Streptococcus pneumoniae, Vibrio spp., and Yersinia pestis. These bacteria may cause disease in man or animals or plants. Methods of the invention may by used with any bacterium that is capable of binding to a lipid bilayer, for example capable of binding to a red blood cell.

The method may usefully be performed on a first microorganism and on a second microorganism, wherein the second microorganism differs from the first microorganism substantially only in relation to a component involved or considered to be involved in adhesion of the microorganism to a lipid bilayer. For example, the first microorganism may be capable of adhering to the lipid bilayer, whilst the second microorganism may be incapable of adhering to the lipid bilayer, or may adhere only under certain circumstances, for example if brought into intimate contact with the lipid bilayer by centrifugation. Comparison of gene expression in the first and second microorganism may reveal differences in gene expression (in addition to the difference relating to the said component) between the first and second microorganism which may be useful, as discussed further below.

As an example, the first microorganism may be an EPEC wild-type in relation to EspA and the second microorganism may be an EPEC that differs from the first microorganism substantially only in relation to EspA. Examples of such microorganisms are given in Example 1. Alternatively, the first microorganism may be an EPEC wild-type for EspB and the second microorganism may be an EPEC that differs from the first test organism substantially only in relation to EspB. The first and second microorganisms may differ in relation to EspC (a type III secretion mutant), EspD, tir or eae.

A further aspect of the invention provides a kit of parts comprising a lipid bilayer substantially not associated with protein or RNA synthetic machinery (for example red blood cells or membranes therefrom) and a nucleic acid microarray and/or. protein microarray. The kit may further comprise other components useful in carrying out a method of the invention, for example components useful in RNA and/or cDNA isolation/preparation and/or protein isolation.

A further aspect of the invention provides a method of identifying a gene of a microorganism, the expression of which differs in the presence or absence of contact and/or adhesion of the microorganism to a lipid bilayer, the method comprising performing the method of any one the preceding claims, and further comprising the step of comparing the expression of at least one gene in the presence and absence of said contact and/or adhesion, and selecting a gene whose expression is different in the presence and absence of after contact and/or adhesion of the microorganism to a lipid bilayer.

A further aspect of the invention provides a method of selecting a target for development or identification of an antiinfective agent or vaccine, wherein a method according to the preceding aspect of the invention is performed and a product of a gene whose expression is identified as differing in the presence and absence of contact and/or adhesion is selected as a target.

The gene product may be used as a target in drug screens or modelling/rational drug design, as well known to those skilled in the art. Thus, screens may be performed to identify/design compounds that are capable of binding to the gene product, or to identify other cellular components with which the identified gene product interacts. The compound may be a drug-like compound or lead compound for the development of a drug-like compound. The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may have a molecular weight of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

The gene product (or fragment, variant or derivative thereof that is capable of provoking an immune response that reacts to a microorganism expressing the gene product) may be useful as a vaccine. A polynucleotide expressing the gene product (or a said fragment, variant or derivative thereof) may be useful as a DNA vaccine, as known to those skilled in the art. A microorganism (killed or alive; preferably an attenuated or non-virulent microorganism) expressing the gene product (or a said fragment, variant or derivative thereof) may be useful as a vaccine.

Polypeptides with a sequence taken from the gene product (or variant thereof) in which one or more of the amino acid residues are chemically modified, before or after the polypeptide is synthesised, may be used providing that the function of the polypeptide, namely the production of a specific immune response in vivo, remains substantially unchanged. Such modifications include forming salts with acids or bases, especially physiologically acceptable organic or inorganic acids and bases, forming an ester or amide of a terminal carboxyl group, and attaching amino acid protecting groups such-as N-t-butoxycarbonyl. Such modifications may protect the polypeptide from in vivo metabolism.

The polypeptide sequence(s) taken from the gene product (or variant thereof) (which preferably form an epitope(s)) may be present as single copies or as multiples, for example tandem repeats. Such tandem or multiple repeats may be sufficiently antigenic themselves to obviate the use of a carrier. It may be advantageous for the polypeptide to be formed as a loop, with the N-terminal and C-terminal ends joined together, or to add one or more Cys residues to an end to increase antigenicity and/or to allow disulphide bonds to be formed. If the polypeptide with the said amino acid sequence, is covalently linked to a carrier, preferably a polypeptide, then the arrangement is preferably such that the putative epitope-forming amino acid sequence forms a loop.

According to current immunological theories, a carrier function should be present in any immunogenic formulation in order to stimulate, or enhance stimulation of, the immune system. It is thought that the best carriers embody (or, together with the antigen, create) a T-cell epitope. The polypeptide sequence from the identified gene product may be associated, for example by cross-linking, with a separate carrier, such as serum albumins, myoglobins, bacterial toxoids and keyhole limpet haemocyanin. More recently developed carriers which induce T-cell help in the immune response include the hepatitis-B core antigen (also called the nucleocapsid protein), presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both.

Alternatively, several copies of the same or different polypeptide may be cross-linked to one another; in this situation there is no separate carrier as such, but a carrier function may be provided by such cross-linking. Suitable cross-linking agents include those listed as such in the Sigrna and Pierce catalogues, for example glutaraldehyde, carbodiimide and succinimidyl 4-(N-maleimidomethyl)cyclohexane-1-carboxylate, the latter agent exploiting the —SH group on the C-terminal cysteine residue (if present). Any of the conventional ways of cross-linking polypeptides may be used, such as those generally described in O'Sullivan et al Anal. Biochem. (1979) 100, 100-108. For example, the first portion may be enriched with thiol groups and the second portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds.

Further useful cross-linking agents include S-acetylthioglycolic acid N-hydroxysuccinimide ester (SATA) which is a thiolating reagent for primary amines which allows deprotection of the sulphydryl group under mild conditions (Julian et al (1983) Anal. Biochem. 132, 68), dimethylsuberimidate dihydrochloride and N,N′-o-phenylenedimaleimide.

If the polypeptide is prepared by expression of a suitable nucleotide sequence in a suitable host, then it may be advantageous to express the polypeptide as a fusion product with a peptide sequence which acts as a carrier. Kabigen's “Ecosec” system is an example of such an arrangement.

The epitope(s) of the invention may be linked to other antigens to provide a dual effect.

A peptidomimetic compound may be used in place of a polypeptide. The term “peptidomimetic” refers to a compound that mimics the conformation and desirable features of a particular peptide as a therapeutic agent, but that avoids the undesirable features. For example, morphine is a compound which can be orally administered, and which is a peptidomimetic of the peptide endorphin.

Therapeutic applications involving peptides are limited, due to lack of oral bioavailability and to proteolytic degradation. Typically, for example, peptides are rapidly degraded in vivo by exo- and endopeptidases, resulting in generally very short biological half-lives. Another deficiency of peptides as potential therapeutic agents is their lack of bioavailability via oral administration. Degradation of the peptides by proteolytic enzymes in the gastrointestinal tract is likely to be an important contributing factor. The problem is, however, more complicated because it has been recognised that even small, cyclic peptides which are not subject to rapid metabolite inactivation nevertheless exhibit poor oral bioavailability. This is likely to be due to poor transport across the intestinal membrane and rapid clearance from the blood by hepatic extraction and subsequent excretion into the intestine. These observations suggest that multiple amide bonds may interfere with oral bioavailability. It is thought that the peptide bonds linking the amino acid residues in the peptide chain may break apart when the peptide drug is orally administered.

There are a number of different approaches to the design and synthesis of peptidomimetics. In one approach, such as disclosed by Sherman and Spatola, J. Am. Chem. Soc., 112: 433 (1990), one or more amide bonds have been replaced in an essentially isoteric manner by a variety of chemical functional groups. This stepwise approach has met with some success in that active analogues have been obtained. In some instances, these analogues have been shown to possess longer biological half-lives than their naturally-occurring counterparts. Nevertheless, this approach has limitations. Successful replacement of more than one amide bond has been rare. Consequently, the resulting analogues have remained susceptible to enzymatic inactivation elsewhere in the molecule. When replacing the peptide bond it is preferred that the new linker moiety has substantially the same charge distribution and substantially the same planarity as a peptide bond.

Retro-inverso peptidomimetics, in which the peptide bonds are reversed, can be synthesised by methods known in the art, for example such as those described in MθziΠre et al (1997) J. Immunol. 159 3230-3237. This approach involves making pseudopeptides containing changes involving the backbone, and not the orientation of side chains. Retro-inverse peptides, which contain NH—CO bonds instead of CO—NH peptide bonds, are much more resistant to proteolysis.

In another approach, a variety of uncoded or modified amino acids such as D-amino acids and N-methyl amino acids have been used to modify mammalian peptides. Alternatively, a presumed bioactive conformation has been stabilised by a covalent modification, such as cyclisation or by incorporation of γ-lactam or other types of bridges. See, eg. Veber et al, Proc. Natl. Acad. Sci. USA, 75:2636 (1978) and Thursell et al, Biochem. Biophys. Res. Comm., 111:166 (1983).

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

One approach to the synthesis of cyclic stabilised peptidomimetics is ring closing metathesis (RCM). This method involves steps of synthesising a peptide precursor and contacting it with a RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

A further aspect of the invention provides a microorganism in which a gene identified using a method according the previous aspect of the invention is mutated or overexpressed. It is preferred that the mutation in the gene is a deletion or a frameshift mutation or any other mutation that is substantially incapable of reverting. Such gene-specific mutations can be made using standard procedures such as introducing into the microorganism a copy of the mutant gene on an autonomous replicon (such as a plasmid or viral genome) and relying on homologous recombination to introduce the mutation into the copy of the gene in the genome of the microorganism. The mutant microorganism may be useful in determining the role of the identified gene in adhesion to lipid bilayers/host cells and may therefore be useful in determining whether the identified gene or its product is likely to be a useful target for intervention to reduce adhesion of the microorganism to host cells.

A further aspect of the invention provides a gene identified using a said method. A further aspect of the invention provides a polypeptide encoded by the said gene.

By “gene” we include not only the regions of DNA that code for a polypeptide but also regulatory regions of DNA such as regions of DNA that regulate transcription, translation and, for some microorganisms, splicing of RNA. Thus, the gene may include promoters, transcription terminators, ribosome-binding sequences and for some organisms introns and splice recognition sites.

Typically, sequence information of the gene identified using a said method is derived. Conveniently, sequences close to the end of the known sequence of the gene (for example the sequence of the immobilised nucleic acid for which a change in the intensity of hybridisation is observed) may be used for designing amplification or sequencing primers, or hybridisation probes, as known to those skilled in the art. The sequence may also be used to probe databases of nucleic acid sequences, such as the EMBL database, as known to those skilled in the art, in order to identify related or neighbouring sequences.

It may be preferred that hybridisation probing is done under stringent conditions to ensure that the gene, and not a relative, is obtained. By “stringent” is meant that the gene hybridises to the probe when the gene is immobilised on a membrane and the probe (which, in this case is >200 nucleotides in legnth) is in solution and the immobilised gene/hybridised probe is washed in 0.1×SSC at 65° C. for 10 mins. SSC is 0.15 M NaCl/0.015M Na citrate. If seeking to obtain the equivalent gene from a different (preferably related) microorganism, it may be appropriate to use a lower degree of stringency.

The isolated gene can be expressed in a suitable host cell. Thus, the gene (DNA) may be used in accordance with known techniques, appropriately modified in view of the teachings contained herein, to construct an expression vecto, which is then used to transform an appropriate host cell for the expression and production of the polypeptide of the invention. Such techniques include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The DNA encoding the polypeptide of the invention may be joined to a wide variety of other DNA sequences for, introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. Thus, the DNA insert may be operatively linked to an appropriate promoter. Bacterial promoters include the E.coli lacI and lacZ promoters, the T3 and T7 promoters, the gpt promoter, the phage λ PR and PL promoters, the phoA promoter and the trp promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome binding site for translation. (Hastings et al, International Patent No. WO 98/16643, published 23 Apr. 1998)

The vector is then introduced into the host through standard techniques. Generally, not all of the hosts will be transformed by the vector and it will therefore be necessary to select for transformed host cells. One selection technique involves incorporating into the expression vector a DNA sequence marker, with any necessary control elements, that codes for a selectable trait in the transformed cell. These markers include dihydrofolate reductase, G418 or neomycin resistance for eukaryotic cell culture, and tetracyclin, kanamycin or ampicillin resistance genes for culturing in E.coli and other bacteria. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

The polypeptide of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography (for example using a tag fused to the polypeptide), hydroxylapatite chromatography and lectin chromatography. Most preferably, high performance liquid chromatography (“HPLC”) is employed for purification.

Many expression systems are known, including systems employing: bacteria (eg. E.coli and Bacillus subtilis) transformed with, for example, recombinant bacteriophage, plasmid or cosmid DNA expression vectors; yeasts. (eg. Saccaromyces cerevisiae) transformed with, for example, yeast expression vectors; insect cell systems transformed with, for example, viral expression vectors (eg. baculovirus); plant cell systems transfected with, for example viral or bacterial expression vectors; animal cell systems transfected with, for example, adenovirus expression vectors.

The vectors include a prokaryotic replicon, such as the Col E1 ori, for propagation in a prokaryote, even if the vector is to be used for expression in other, non-prokaryotic cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors containing the coding sequence and, for example appropriate transcriptional or translational controls. One such method involves ligation via homopolymer tails. Another method involves ligation via cohesive ends. A further method uses synthetic molecules called linkers and adaptors. Synthetic linkers containing a variety of restriction endonuclease sites are commercially available from a number of sources including International Biotechnologies Inc, New Haven, Conn., USA. A desirable way to modify the DNA encoding the polypeptide of the invention is to use the polymerase chain reaction as disclosed by Saiki et al (1988) Science 239, 487-491. In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

Variants of the genes also form part of the invention. It is preferred if the variant has at least 70% sequence identity, more preferably at least 85% sequence identity, most preferably at least 95% sequence identity with the genes identified by the method of the invention. Of course, replacements, deletions and insertions may be tolerated. The degree of similarity between one nucleic acid sequence and another can be determined using the GAP program of the University of Wisconsin Computer Group. Similarly, variants of proteins encoded by the genes are included.

By “variants” we include insertions, deletions and substitutions, either conservative or non-conservative, where such changes do not substantially alter the normal function of the protein. Fragments are also included, for example fragments that may be useful in raising an immune response that recognises the polypeptide, for example as expressed in the microorganism, even if the fragment does not retain the normal function of the protein.

By Aconservative substitutions” is intended combinations such as Gly, Ala; Val, Ile, Leu; Asp, Glu; Asn, Gln; Ser, Thr; Lys, Arg; and Phe, Tyr. Mutations may be made using the methods of protein engineering and site-directed mutagenesis as well known to those skilled in the art.

A further aspect of the invention provides a method of identifying a compound which reduces the ability of a microorganism to adhere to a host cell comprising the step of selecting a compound which interferes with the function of the said gene or polypeptide encoded by the said gene.

Pairwise screens for compounds which affect the wild type microorganism cell. but not a microorganism cell overexpressing a gene identified by a method of the invention form part of this aspect of the invention.

For example, in one embodiment one cell is a wild type EPEC cell and a second cell is a EPEC cell that is made to overexpress a gene identified and/or isolated by a method of the invention. The ability of each cell to adhere to a lipid bilayer, for example red blood cell, is determined in the presence of a compound to be tested, to identify which compound reduces adhesion by the wild type cell but not the cell overexpressing the said gene. The screen may alternatively be performed using cells such as epithelial cells in place of red blood cells or other lipid bilayer that is substantially not associated with protein or RNA synthetic machinery. Methods of determining the extent of adherence will be apparent to those skilled in the art; for example, the extent of haemolysis of red blood cells may be measured, as described in Example 1. Thus, the measurement of adherence need not be direct, ie an outcome dependent on adherence may be measured.

Pairwise screens and other screens for compound are generally disclosed in Kirsh & Di Domenico (1993) in “The Discovery of Natural Products with a Therapeutic Potential” (Ed, V. P. Gallo), Chapter 6, pages 177-221, Butterworths, V K (incorporated herein by reference).

A further aspect of the invention provides a molecule which selectively interacts with, and substantially inhibits the function of, the said gene or nucleic acid product thereof, or polypeptide encoded by the said gene. The molecule may be an antisense nucleic acid or nucleic acid derivative. It may be an antisense oligonucleotide or a ribozyme.

By “nucleic acid product thereof” we include any RNA, especially mRNA, transcribed from the gene.

Antisense oligonucleotides are single-stranded nucleic acid, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise sequence-specific molecules which specifically bind double-stranded DNA via recognition of major groove hydrogen binding sites.

Clearly, the sequence of the antisense nucleic acid or oligonucleotide can readily be determined by reference to the nucleotide sequence of the gene in question. By binding to the target nucleic acid, the above oligonucleotides can inhibit the function of the target nucleic acid. This could, for example, be a result of blocking the transcription, processing, poly(A)addition (if appropriate), replication, translation, or promoting inhibitory mechanisms of the cells, such as promoting RNA degradations.

Oligonucleotides are subject to being degraded or inactivated by cellular endogenous nucleases. To counter this problem, it is possible to use modified oligonucleotides, eg having altered intemucleotide linkages, in which the naturally occurring phosphodiester linkages have been replaced with another linkage. For example, Agrawal et al (1988) Proc. Natl. Acad. Sci. USA 85, 7079-7083 showed increased inhibition in tissue culture of HIV-1 using oligonucleotide phosphoramidates and phosphorothioates. Sarin et al (1988) Proc. Natl. Acad. Sci. USA 85, 7448-7451 demonstrated increased inhibition of HIV-1 using oligonucleotide methylphosphonates. Agrawal et al (1989) Proc. Natl. Acad. Sci. USA 86, 7790-7794 showed inhibition of HIV-1 replication in both early-infected and chronically infected cell cultures, using nucleotide sequence-specific oligonucleotide phosphorothioates. Leither et al (1990) Proc. Natl. Acad. Sci. USA 87, 3430-3434 report inhibition in tissue culture of influenza virus replication by oligonucleotide phosphorothioates.

Oligonucleotides having artificial linkages have been shown to be resistant to degradation in vivo. For example, Shaw et al (1991) in Nucleic Acids Res. 19, 747-750, report that otherwise unmodified oligonucleotides become more resistant to nucleases in vivo when they are blocked at the 3N end by certain capping structures and that uncapped oligonucleotide phosphorothioates are not degraded in vivo.

A detailed description of the H-phosphonate approach to synthesizing oligonucleoside phosphorothioates is provided in Agrawal and Tang (1990) Tetrahedron Letters 31, 7541-7544, the teachings of which are hereby incorporated herein by reference. Syntheses of oligonucleoside methylphosphonates, phosphorodithioates, phosphoramidates, phosphate esters, bridged phosphoramidates and bridge phosphorothioates are known in the art. See, for example, Agrawal and Goodchild (1987) Tetrahedron Letters 28, 3539; Nielsen et al (1988) Tetrahedron Letters 29, 2911; Jager et al (1988) Biochemistry 27, 7237; Uznanski et al (1987) Tetrahedron Letters 28, 3401; Bannwarth (1988) Helv. Chim. Acta. 71, 1517; Crosstick and Vyle (1989) Tetrahedron Letters 30, 4693; Agrawal et al (1990) Proc. Natl. Acad. Sci. USA 87, 1401-1405, the teachings of which are incorporated herein by reference. Other methods for synthesis or production also are possible. In a preferred embodiment the oligonucleotide is a deoxyribonucleic acid (DNA), although ribonucleic acid (RNA) sequences may also be synthesized and applied.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridization and are less likely to be effective, relative to their full-length counterparts. Thus, it is desirable to use oligonucleotides that are resistant to degradation in the body and which are able to reach the targeted cells. The present oligonucleotides can be rendered more resistant to degradation in vivo by substituting one or more internal artificial intemucleotide linkages for the native phosphodiester linkages, for example, by replacing phosphate with sulphur in the linkage. Examples of linkages that may be used include phosphorothioates, methylphosphonates, sulphone, sulphate, ketyl, phosphorodithioates, various phosphoramidates, phosphate esters, bridged phosphorothioates and bridged phosphoramidates. Such examples are illustrative, rather than limiting, since other intemucleotide linkages are known in the art. See, for example, Cohen, (1990) Trends in Biotechnology. The synthesis of oligonucleotides having one or more of these linkages substituted for the phosphodiester internucleotide linkages is well known in the art, including synthetic pathways for producing oligonucleotides having mixed intemucleotide linkages.

Oligonucleotides can be made resistant to extension by endogenous enzymes by Acapping≅ or incorporating similar groups on the 5N or 3N terminal nucleotides. A reagent for capping is commercially available as Amino-Link II™ from Applied BioSystems Inc, Foster City, Calif. Methods for capping are described, for example, by Shaw et al (1991) Nucleic Acids Res. 19, 747-750 and Agrawal et al (1991) Proc. Natl. Acad. Sci. USA 88(17), 7595-7599, the teachings of which are hereby incorporated herein by reference.

A further method of making oligonucleotides resistant to nuclease attack is for them to be “self-stabilized” as described by Tang et al (1993) Nuc. Acids Res. 21, 2729-2735 incorporated herein by reference. Self-stabilized oligonucleotides have hairpin loop structures at their 3′ ends, and show increased resistance to degradation by snake venom phosphodiesterase, DNA polymerase I and fetal bovine serum. The self-stabilized region of the oligonucleotide does not interfere in hybridization with complementary nucleic acids, and pharmacokinetic and stability studies in mice have shown increased in vivo persistence of self-stabilized oligonucleotides with respect to their linear counterparts.

In accordance with the invention, the inherent binding specificity of antisense oligonucleotides characteristic of base pairing is enhanced by limiting the availability of the antisense compound to its intended locus in vivo, permitting lower dosages to be used and minimizing systemic effects. Thus, oligonucleotides are applied locally to achieve the desired effect. The concentration of the oligonucleotides at the desired locus is much higher than if the oligonucleotides were administered systemically, and the therapeutic effect can be achieved using a significantly lower total amount. The local high concentration of oligonucleotides enhances penetration of the targeted cells and effectively blocks translation of the target nucleic acid sequences.

The oligonucleotides can be delivered to the locus by any means appropriate for localized administration of a drug. For example, a solution of the oligonucleotides can be injected directly to the site or can be delivered by infusion using an infusion pump. The oligonucleotides also can be incorporated into an implantable device which when placed at the desired site, permits the oligonucleotides to be released into the surrounding locus.

The oligonucleotides may be administered via a hydrogel material. The hydrogel is noninflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10 to about 80% by weight ethylene oxide and from about 20 to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic^{R}.

In this embodiment, the hydrogel is cooled to a liquid state and the oligonucleotides are admixed into the liquid to a concentration of about 1 mg oligonucleotide per gram of hydrogel. The resulting mixture then is applied onto the surface to be treated, for example by spraying or painting during surgery or using a catheter or endoscopic procedures. As the polymer warms, it solidifies to form a gel, and the oligonucleotides diffuse out of the gel into the surrounding cells over a period of time defined by the exact composition of the gel.

The oligonucleotides can be administered by means of other implants that are commercially available or described in the scientific literature, including liposomes, microcapsules and implantable devices. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the oligonucleotides. The oligonucleotides can be incorporated into the material as it is polymerized or solidified, using melt or solvent evaporation techniques, or mechanically mixed with the material. In one embodiment, the oligonucleotides are mixed into or applied onto coatings for implantable devices such as dextran coated silica beads, stents, or catheters. Polymeric nanoparticles/biodegradable drug carriers may also be used (Mader (1998) Radiol. Oncol. 32, 89-94).

The dose of oligonucleotides is dependent on the size of the oligonucleotides and the purpose for which it is administered. In general, the range is calculated based on the surface area of tissue to be treated. The effective dose of oligonucleotide is somewhat dependent on the length and chemical composition of the oligonucleotide but is generally in the range of about 30 to 3000 :g per square centimetre of tissue surface area.

The oligonucleotides may be administered to the patient systemically for both therapeutic and prophylactic purposes. The oligonucleotides may be administered by any effective method, for example, parenterally (eg intravenously, subcutaneously, intramuscularly) or by oral, nasal or other means which permit the oligonucleotides to access and circulate in the patient's bloodstream. Oligonucleotides administered systemically preferably are given in addition to locally administered oligonucleotides,. but also have utility in the absence of local administration. A dosage in the range of from about 0.1 to about 10 grams per administration to an adult human generally will be effective for this purpose.

The molecule may be an antibody that binds selectively to the gene product.

By an antibody is included an antibody or other immunoglobulin, or a fragment or derivative thereof, as discussed further below.

The variable heavy (V_{H}) and variable light (V_{L}) domains of the antibody are involved in antigen recognition, a fact first recognised by early protease digestion experiments. Further confnrmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

Preferably, the antibody has an affinity for the epitope of between about 10⁵.M⁻¹ to about 10¹².M⁻¹, more preferably at least 10⁸.M¹.

Suitable monoclonal antibodies to selected antigens may be prepared by known techniques, for example those disclosed in A Monoclonal Antibodies: A manual of techniques≅, H Zola (CRC Press, 1988) and in A Monoclonal Hybridoma Antibodies: Techniques and Applications≅, J G R Hurrell (CRC Press, 1982). Chimaeric antibodies are discussed by Neuberger et al (1988, 8th International Biotechnology Symposium Part 2, 792-799). Suitably prepared non-human antibodies can be “humanized” in known ways, for example by inserting the CDR regions of mouse antibodies into the framework of human antibodies.

It will be appreciated that the molecules of this aspect of the invention are useful in treating or preventing any infection caused by the microorganism from which the said gene has been isolated, or a close relative of said microorganism. Thus the said molecule is an antiinfective.

A further aspect of the invention provides a compound or molecule identified or identifiable by the method of the preceding aspect of the invention.

A further aspect of the invention provides a molecule or compound or polypeptide of the invention or nucleic acid encoding said polypeptide for use in medicine.

A further aspect of the invention provides the use of a compound, molecule, polypeptide or polynucleotide as defined in relation to the preceding aspect of the invention for treating infection of a host organism with said microorganism. A further aspect of the invention provides a method of treating a host which has, or is susceptible to, an infection with a microorganism, the method comprising administering an effective amount of a molecule, compound, polypeptide or polynucleotide as defined in relation to the previous aspect of the invention to the host, wherein said gene is present in said microorganism, or a close relative of said microorganism. By “effective amount” we mean an amount which substantially prevents or ameliorates the infection. By “host” we include any animal which may be infected by a microorganism.

A further aspect of the invention provides a method of treating a host which has, or is susceptible to, an infection with a microorganism, the method comprising administering an effective amount of a molecule or compound of the invention, wherein said gene is present in said microorganism, or a close relative of said microorganism.

A further aspect of the invention provides the use of a gene or polypeptide of the invention or microorganism overexpressing same in the manufacture of a medicament for vaccination of a host which has or is susceptible to, an infection with a microorganism, wherein said gene is present in said microorganism, or a close relative of said microorganism.

A further aspect of the invention provides a pharmaceutical composition comprising a molecule or compound or polypeptide or polynucleotide of the invention and a pharmaceutically acceptable carrier.

If the composition is intended for use as a vaccine, it may be appropriate to include an adjuvant. Suitable adjuvants include Freund's complete or incomplete adjuvant, muramyl dipeptide, the “Iscoms” of EP 109 942, EP 180 564 and EP 231 039, aluminium hydroxide, saponin, DEAE-dextran, neutral oils (such as miglyol), vegetable oils (such as arachis oil), liposomes, Pluronic polyols or the Ribi adjuvant system (see, for example GB-A-2 189 141). “Pluronic” is a Registered Trade Mark.

The aforementioned compounds or a formulation thereof may be administered by any conventional method including oral and parenteral (eg subcutaneous or intramuscular) injection. The treatment may consist of a single dose or a plurality of doses over a period of time.

Whilst it is possible for a compound to be administered alone, it is preferable to present it as a pharmaceutical formulation, together with one or more acceptable carriers. The carrier(s) must be “acceptable” in the sense of being compatible with the compound and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

It should be understood that in addition to the ingredients particularly mentioned above the formulations of this invention may include other agents conventional in the art having regard to the type of formulation in question, for example those suitable for oral administration may include flavouring agents.

The formulation may be suitable for use as a food supplement or as an additive of food. The food may be a milk substitute. Preferably, the food is suitable for administration to a human baby or infant or a young animal. Howeve, it may be suitable for any individual which is susceptible to infection, for example bacterial infection, including older animals. Exemplary animals include domestic cattle, especially calves; and poultry such as chickens and turkeys. A further aspect of the invention provides a food product comprising a foodstuff and an active ingredient as discussed above. The foodstuff may be adapted for consumption by animals.

A gene or gene product identified using a method of the invention may also be useful in diagnosis. For example, the gene or gene product may be characteristic of a particular microorganism or grouping of microorganisms. Checking for the presence of the gene or of its expression may therefore be useful in determining whether that microorganism is present (for example in a sample from a patient) and/or in determining a suitable treatment for a patient. The presence of the gene or its expression, or its expression product may be detected using techniques adapted from those well known to those skilled in the art. For example antibodies or fragments that bind specifically to the gene product may be used, and/or PCR-based techniques for detecting the presence of the gene, and optionally its expression, may be used. Gene expression may also be detected by DNA microarray, as discussed above and the gene products may also be detected/identified using other techniques known to those skilled in the art, including protein arrays, FPLC/HPLC, mass spectrometry, NMR or calorimetric reaction.

A further aspect of the invention therefore provides a method of microorganism, for example bacterial, detection and/or characterisation wherein the presence/absence and/or expression of a gene identifiable by a method of the invention in a sample is determined.

The presence of expression of a said gene may be determined by determination of the presence of a phenotypic characteristic in a sample. The methods of the invention may allow the identification of a metabolic pathway/phenotype that is characteristic of a particular microorganism or grouping of microorganisms. The presence/absence of microorganisms possessing this metabolic pathway/phenotype may be detected in a sample by culturing the sample in specific growth conditions/media, using techniques adapted from those well known to those skilled in the art.

The invention is now further described by reference to the following, non-limiting, Figures and Examples.

### FIGURE LEGENDS

FIG. 1. Haemolytic activity due to EPEC and EHEC strains. 1A. EPEC strain E2348/69 and EHEC strain 85-170 were assayed for their ability to haemolyse human RBCs as described in Methods; 1B. Osmoprotection of haemolysis activity was assessed by performing assays with E2348/69 and 85-170 in the presence of 30 mM of the different sugars. 1C. Haemolytic activity due to wildtype and plasmid-cured EPEC E2348/69, deletion mutants and complemented mutant strains.

FIG. 2. Phase contrast micrographs showing an uninfected RBC monolayer (A), RBC monolayers infected with EPEC strain E2348/69 (B), EHEC strain 85-170 (C), and EPEC deletion mutant strains UMD872(ΔespA) (D), UMD864(ΔespB) (E) and UMD870(ΔespD) (F). Wildtype EPEC and EHEC strains were highly haemolytic whereas strain UMD864 showed reduced haemolysis. Strains UMD872 and UMD870 did not adhere to RBCs and were non-haemolytic. Magnification bar, 5 μm.

FIG. 3. Adhesion of EPEC strains to RBC monolayers. Strains lacking intimin (CVD206) and Bfp (JPN15) adhered to RBCs as effectively as wildtype E2348/69 whereas a strain lacking EspA (UM872) was non-adherent.

FIG. 4. Scanning (SEM) (A-C, E-G) and transmission (TEM) electron micrographs (D) showing attachment of EPEC and EHEC to RBC monolayers. Large filamentous structures promoted attachment of EPEC strain E2348/69 to intact and lysed RBCs (A, B, arrows) and these structures were confirmed as EspA filaments by immunofluorescence (A, inset) and by immunogold labelling (C, D); filaments coated with gold particles were seen by both SEM and TEM (C, D, arrows). Note the ˜50 nm part of the EspA filament structure close to the bacterial surface which did not stain with the EspA antibody (D, inset, arrowheads). Identical filaments promoted attachment of strains UMD864(ΔespB) (E, arrow) and 85-170 (G, arrow) whereas strain UMD872(pICC25) showed a more intimate attachment but no detectable EspA filaments (F). Magnification bars: A, 1 μm; B-G 0.2 μm; D, inset 0.1 μm.

FIG. 5. Analysis of EPEC secreted proteins associated with RBC membranes following haemolysis. Following infection, purified RBC membranes were separated by SDS-PAGE, and EspD detected using monoclonal EspD antiserum (arrow). EspD was found associated with RBC membranes infected with E3248/69 (Lane 2), but not with membranes infected with UMD872 (Lane 1). Molecular weight markers (KDa1) are indicated.

FIG. 6. Immunofluorescence of EPEC secreted proteins following haemolysis. A weak but distinct punctate staining of EspD (A, arrow) was seen in E2348/69 infected RBC membranes but no staining of EspB (B). Magnification bar, 5 μm.

FIG. 7. Scanning electron micrograph (SEM) of Salmonella typhimurium bound to RBCs.

FIG. 8. SEM of Heliobacter pylori bound to RBCs. Binding of H. pylori to RBCs induced specific expression of a subset of genes not expressed in the control (no RBCs present).

### EXAMPLE 1

### EspA Filament-Mediated Protein Translocation into Red Blood Cells

Type III secretion allows bacteria to inject effector proteins into host cells. In enteropathogenic E. coli (EPEC), three type III secreted proteins, EspA, EspB and EspD have been shown to be required for translocation of the Tir effector protein into host cells. EspB and EspD have been proposed to form a pore in the host cell membrane whereas EspA, which forms a large filamentous structure bridging bacterial and host cell surfaces, is thought to provide a conduit for translocation of effector proteins between pores in the bacterial and host cell membranes. Type III secretion has been correlated with an ability to cause contact-dependent haemolysis of red blood cells (RBC) in vitro. Since EspA filaments link bacteria and the host cell, it was possible that intimate bacterial-RBC contact would not be required for EPEC-induced haemolysis. We investigated the interaction of EPEC with monolayers of RBC attached to polylysine coated cell culture dishes. EPEC caused total RBC haemolysis in the absence of centrifugation and osmoprotection studies were consistent with the insertion of a hydrophilic pore into the RBC membrane. Cell attachment and haemolysis involved interaction between EspA filaments and the RBC membrane and was dependent upon a functional type III secretion system and on EspD whereas EPEC lacking EspB still caused some haemolysis. Following haemolysis, only EspD was consistently detected in the RBC membrane. This Example shows that intimate bacterial-RBC membrane contact is not a requirement for EPEC-induced haemolysis and it also provides further evidence that EspA filaments are a conduit for protein translocation and that EspD may be the major component of a translocation pore in the host cell membrane.

Similar interactions were also found when RBCs were infected with Helicobacter pylori.

Results

EPEC-Induced Haemolysis does not Require Intimate Bacterial-RBC Membrane Contact.

Yersinia and Shigella induced haemolysis requires centrifugation of bacteria and RBCs in order to reduce the distance between bacterial and red cell membranes below a critical threshold. This has been termed contact-dependent haemolysis (Clerc et al 1986; Hakansson et al 1996) and contact-induced haemolysis was recently demonstrated with EPEC (Warawa et al 1999). However, since long

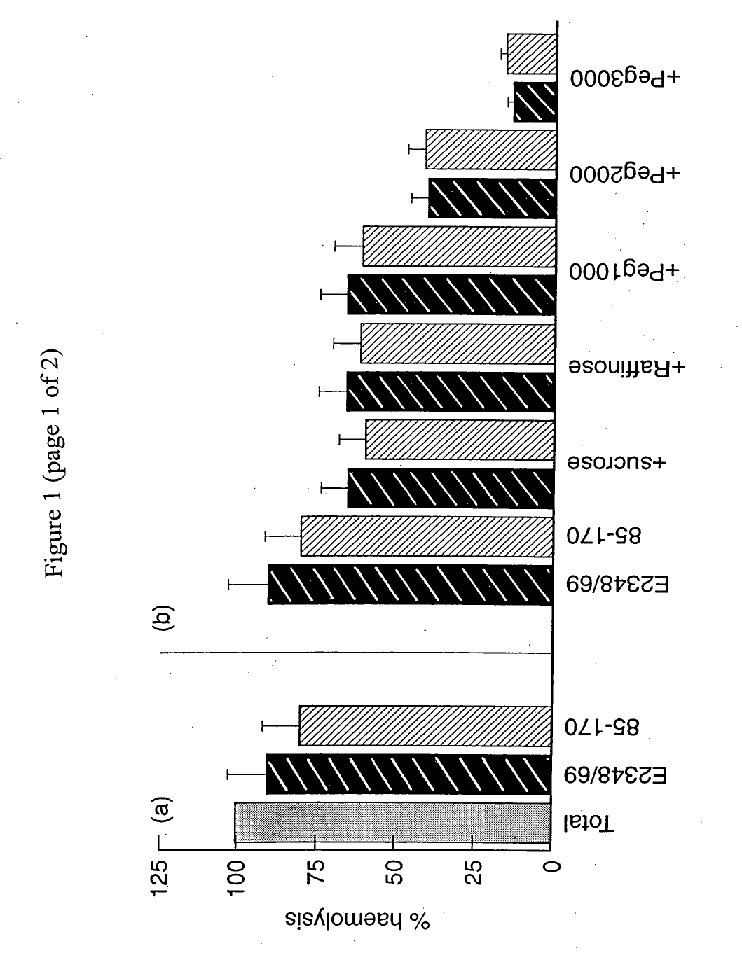

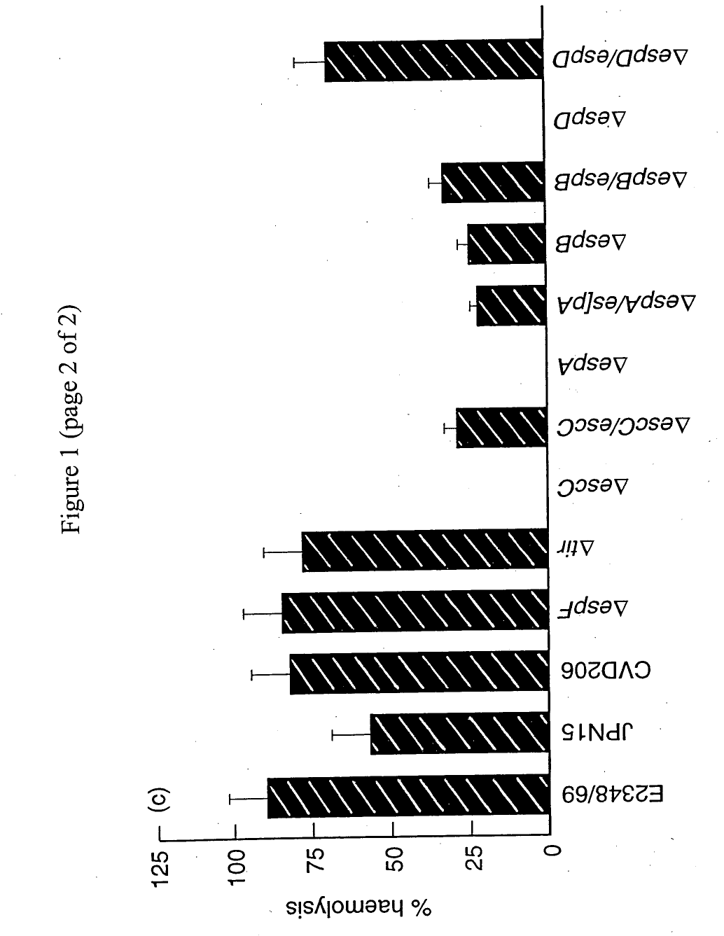

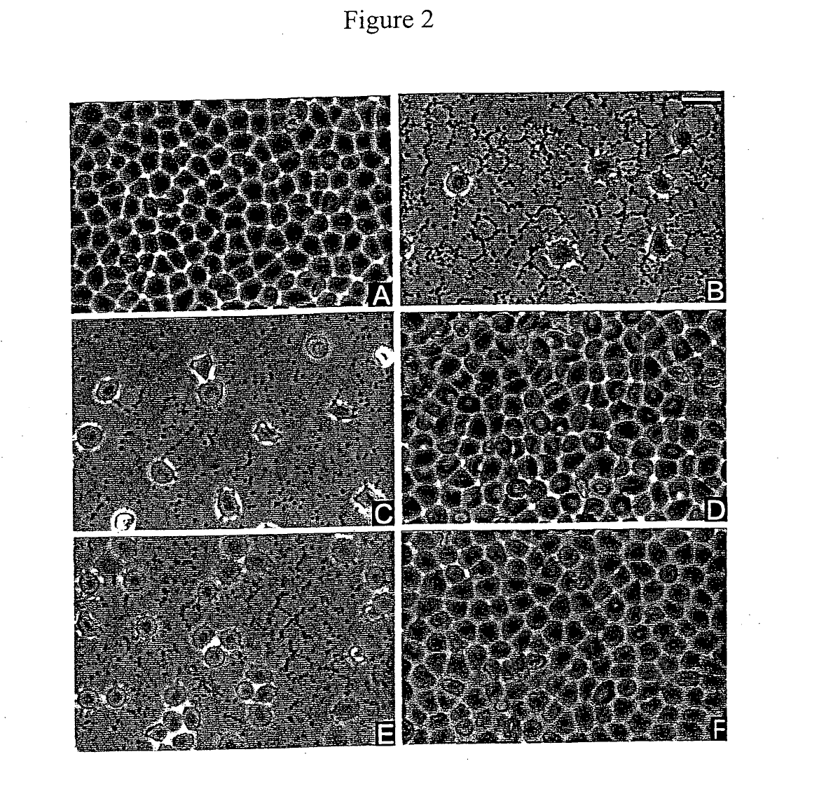

EspA filaments were shown to connect bacteria to the host cell during protein translocation (Knutton et al 1998), we predicted that intimate bacterial-RBC contact and thus centrifugation should not be required for EPEC-induced haemolysis. We therefore developed an haemolysis assay equivalent to typical cell culture adhesion assays by using monolayers of RBCs attached to cell culture dishes. Wildtype EPEC strain E2348/69 was incubated with RBC monolayers for up to 6 h at 37° C. and release of haemoglobin was monitored as described in Methods. Haemoglobin release was negligible after 1 h but then increased with time and was maximal (>90% haemolysis) after 4 h (FIG. 1A, 2). Attachment to polylysine coated plates did not contribute to haemolysis since similar levels of haemolysis occurred in the absence of polylysine when RBCs were allowed to settle and attach to cell culture treated dishes. However, the formation of tightly attached RBC monolayers made the assay much easier to perform and improved reproducibility. In this assay system there was no significant additional effect of centrifuging bacteria onto RBC monolayers and haemolysis of human RBCs was not affected by blood group type (A, B, AB, O) (data not shown). Enterohaemorrhagic E. coli (EHEC) strain 85-170 (O157:H7), was also tested and shown to induce a non-contact dependent haemolysis of human RBCs (FIGS. 1A, 2).

Blocker et al (Blocker et al 1999) recently demonstrated that haemolytic activity of Shigella could be prevented by addition of osmotic protectants to the medium, a mechanism consistent with the insertion of a hydrophilic pore into the RBC membrane; addition of molecules too large to pass through a membrane pore counter balance the increased intracellular pressure thereby reducing haemolysis. We performed similar experiments and FIG. 1B shows the effect on haemolysis due to EPEC strain E2348/69 and EHEC strain 85-170 in the presence of different osmoprotectants at a concentration of 30 mM. As was the case with Shigella, molecules larger then PEG1000 yielded significant protection against lysis and protection increased with the size of the molecule.

Role of the Type III Secretion System, Type III Secreted Proteins and other Virulence Proteins in EPEC-Induced Haemolysis.

An association of the EPEC type III secretion system with haemolytic activity was confirmed using a type III secretion mutant (EscC) of E2348/69; no significant haemoglobin release was detected with this strain (FIG. 1C). The same assay was used to assess the involvement in haemolysis of EPEC type III secreted proteins. EPEC with single mutations in genes encoding secreted proteins were examined and, as previously reported using a contact-dependent haemolysis assay (Warawa et al 1999), tir and espF mutants induced levels of haemolysis comparable to the wild-type strain whereas no lysis was observed with espA and espD mutants. In each case haemolysis was restored, albeit not to levels of the wild type, when the deleted gene was re-introduced on a plasmid (FIG. 1C). Interestingly, an espB mutant reproducibly caused some (−25%) haemolysis which increased to ˜40% when the espB gene was re-introduced (FIG. 1C). We have previously shown that an espD mutant makes barely detectable EspA filaments which could theoretically be functional if brought into intimate contact with the host cell membrane. However, centrifugation of this strain onto RBC monolayers did not induce haemolysis (data not shown). EPEC strains lacking the EPEC Adherence Factor (EAF) plasmid (strain JPN15) and intimin (strain CVD206) induced levels of haemolysis comparable to wild-type E2348/69 (FIG. 1C).

We have previously shown that the coiled-coil domain of EspA is important for assembly of functional EspA filaments on the surface of EPEC; disruption of the coiled-coil domain of EspA by a double radical amino acid substitution (strain UMD872(pICC27)) prevented EspA filament formation, protein translocation and A/E lesion formation (Delahay et al 1999). However, a single radical amino acid substitution (strain UMD872(pICC25)) was insufficient to totally disrupt the coiled-coil domain and A/E lesion formation but resulted in greatly shortened EspA filaments similar to those produced by an espD mutant. Both strains were tested for their ability to induce haemolysis. No lysis was observed with strain UMD872(pICC27) whereas strain UMD872(pICC25) did routinely produce low levels (˜10%) of lysis; interestingly, this figure increased to ˜40% when this strain was centrifuged onto the RBC monolayer. Centrifugation of the EspA minus strain (UMD872) onto RBC monolayers did not induce haemolysis.

EspA4 Filamentv Mediate EPEC Binding to RBCs

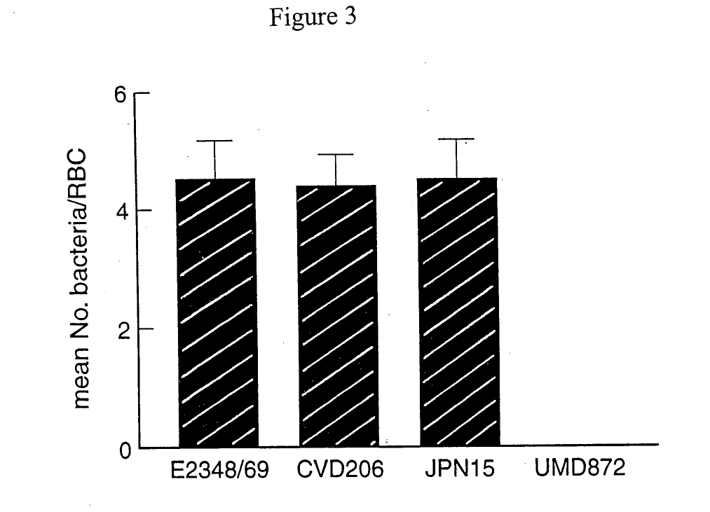

Phase contrast microscopy revealed bacterial attachment to RBCs of all the haemolytic strains (wild type, EAF plasmid mutant, eae mutant, tir mutant, espF mutant, espB mutant) but no adhesion of the non-haemolytic strains (espA mutant, espD mutant); bacterial adhesion was primarily as individual bacteria and not bacterial microcolonies as is typical of adhesion to cultured cells (FIG. 2). The plasmid-encoded bundle forming pilus (Bfp) (Giron et al 1991), intimin and EspA filaments are known to play a role in EPEC adhesion to cultured cells. In order to determine which adhesin was important in EPEC adhesion to RBCs we performed quantitative adhesion assays using wildtype (E2348/69), intimin minus (CVD206), Bfp minus (JPN15) and EspA minus (UMD872) strains; lack of intimin or Bfp had no effect on EPEC adhesion to RBCs whereas lack of EspA filaments resulted in a total loss of bacterial adherence (FIG. 3).

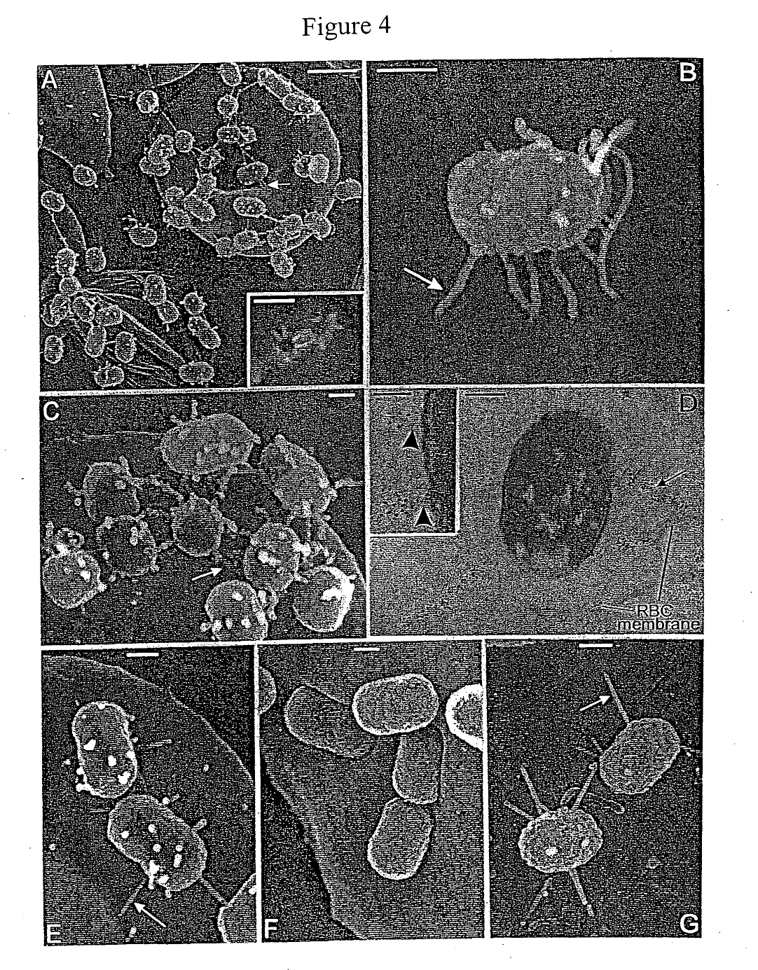

The importance of EspA filaments in RBC adhesion was confirmed by scanning (SEM) and transmission electron microscopy (TEM) which revealed bacterial attachment to both intact and lysed RBCs mediated by large filamentous surface appendages characteristic of EspA filaments; immuno-fluorescence and immunogold labelling confirmed these structures as EspA filaments (FIG. 4). Typical EspA filaments promoted RBC attachment of all the haemolytic strains including the wildtype EPEC (FIG. 4B) and tir, espB (FIG. 4E), and espF mutants whereas the espA and espD mutants did not adhere to RBCs and were nonhaemolytic. Strain UMD872(pICC25), which produces barely detectable EspA filaments and low levels of haemolysis, showed a more intimate attachment of bacteria to the RBC membrane but vestigial EspA filaments detected by immunofluorescence (Delahay et al 1999) were not seen bv scanning electron microscopy (FIG. 4F). RBC attachment of EHEC strain 85-170 was also promoted by morphologically similar filaments (FIG. 4G).

Interestingly, by gold labelling TEM, there appeared to be a short ˜50-nm section of the EspA filament structure adjacent to the bacterial surface that did not label with the EspA antiserum (FIG. 4D, inset).

EspD is Transferred to RBC Membranes.

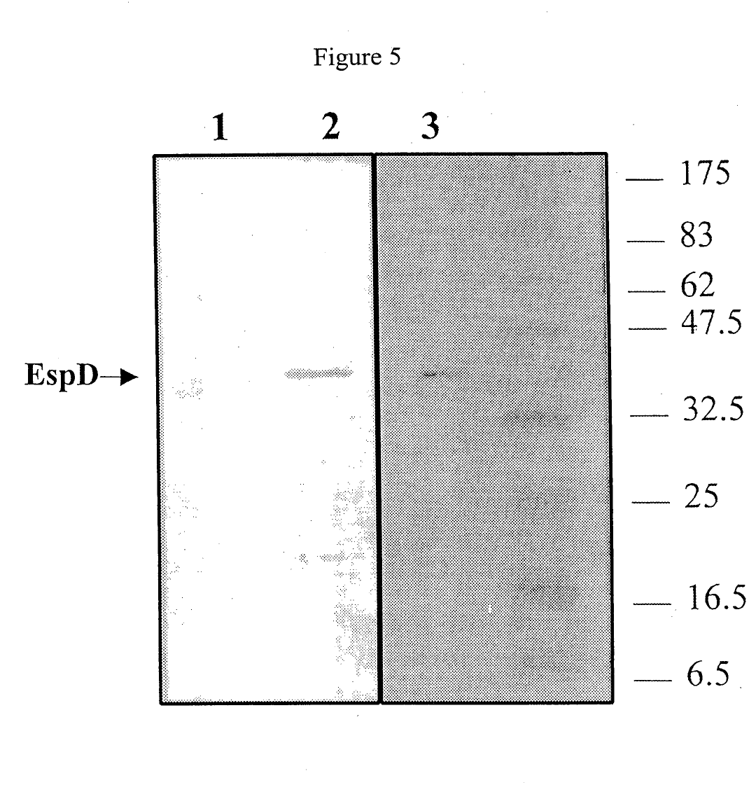

EPEC-induced haemolysis is consistent with insertion of a pore into the red cell membrane. In order to determine which bacterial proteins might become associated with the RBC membrane during haemolysis we isolated RBC membranes following haemolysis by sucrose density gradient centrifugation according to the method of Blocker et al (Blocker et al 1999). Infections were performed using wild-type EPEC (E2348/69), and also strains deficient in EspA (UMD872), EspB (UMD864), and EspD (UMD870). Membrane proteins from each of the infections were separated by SDS-PAGE, blotted and probed with antibodies against EspA, EspB, EspD and Tir. No bacterial proteins were detected in membranes of RBCs infected with UMD872, UMD864 or UMD870. Only EspD was found consistently associated with membranes infected with E2348/69 (FIG. 5); EspB was also detected very faintly, although this result was inconsistent in repeated experiments (data not shown).

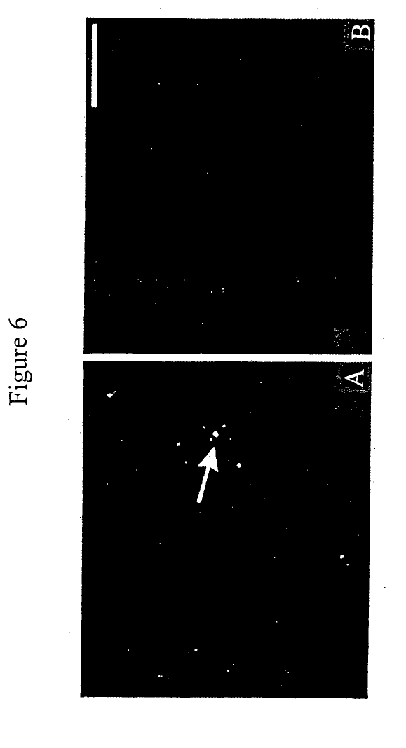

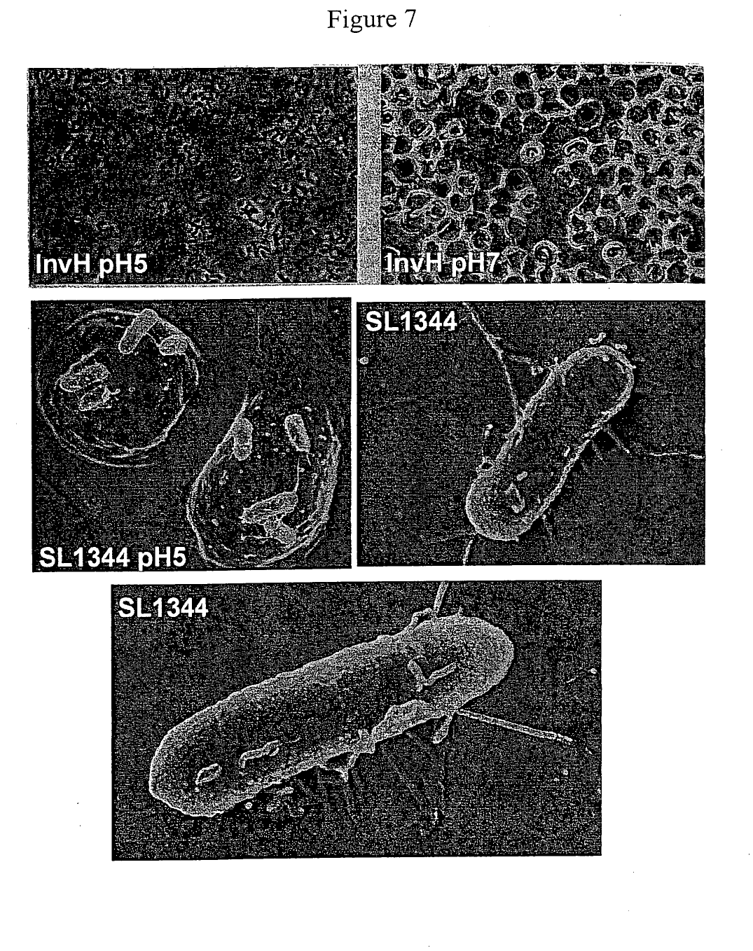

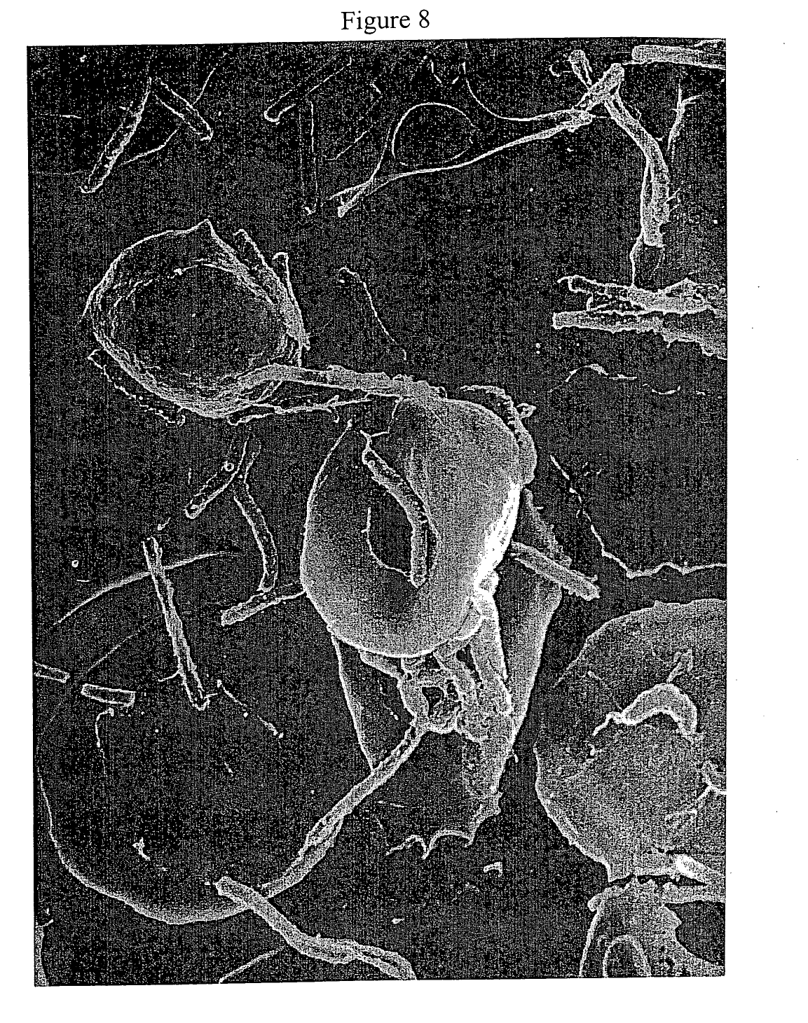

Immuno-fluorescence staining of RBC membranes following infection with E2348/69 revealed no staining with the EspB antibody but a clear punctate staining of some RBC membranes was seen with the EspD antibody (FIG. 6).

Discussion

Using a static infection of RBC monolayers, this study has shown that, unlike the contact dependent haemolysis of Yersinia and Shigella, close bacterial-RBC contact is not a requirement for EPEC or EHEC-induced haemolysis; important for haemolysis, however, was type III secretion, bacterial attachment by EspA filaments, and EspD translocation. The EAF virulence plasmid, intimin, Tir, and EspF do not appear to play a role in haemolytic activity.

Haemolysis of RBC has been correlated with type III protein secretion/translocation although the structural basis for protein translocation has yet to be fully elucidated for any type III secretion system (Hueck, 1998). In EPEC the proposed translocation apparatus (translocon) consists of pores in the bacterial and host cell membrane connected by a hollow EspA filament (Frankel, et al 1998) which would provide a continuous channel from the bacterial to the host cell cytosol. Such a model is consistent with the observed EPEC-induced haemolysis since EspA filaments are essential for haemolysis, EspA filaments mediate interaction of EPEC with the RBC membrane and one putative pore-forming protein, EspD, was localised to the RBC membrane following haemolysis. Involvement of long EspA filaments explains why close bacterial-RBC contact is not a requirement for EPEC-induced haemolysis and contrasts with Yersinia and Shigella which do not possess EspA filaments or related structures. The Shigella secreton consists of a macromolecular complex spanning both bacterial membranes with a 60-nm long external needle structure (Blocker et al 1999). Hence the close Shigella-host cell contact required for protein translocation and close Shigella-RBC contact required for haemolysis. Interestingly, EspA immunogold labelling following haemolysis revealed, in addition to EspA-mediated bacterial attachment to RBCS, a short (˜50-nm) external part of the filament structure which did not stain with the EspA antibody; this could be the equivalent external needle structure of the EPEC type III secreton onto which EspA is polymerised.

Employing non-conservative amino acid substitution, we recently demonstrated the importance of a carboxy terminal EspA coiled-coil domain in EspA filament assembly and function; single substitutions generated mutants defective in filament assembly but which still retained some functional protein translocation and the ability to induce A/E lesion formation whereas double substitutions totally abolished EspA filament assembly, protein translocation and A/E lesion formation (Delahay, et al., 1999). In this study strains with double substitutions were non-haemolytic whereas strains with a single substitution and which produced very short EspA filaments were weakly haemolytic consistent with retained functionality. More interesting however was the observation that haemolysis was increased a further ˜4-fold when this strain was centrifuged onto the RBC monolayer i.e. when very short EspA filaments were brought into intimate contact with the RBC membrane.

Interaction of EHEC strain 85-170 (0157:H7) with RBCs also involved long filamentous structures. Although these filaments could not be confirmed as EspA because they did not react with our EPEC EspA antiserum, they do have all the characteristics of EspA filaments; thus, this is the first description of EspA-like filaments produced by EHEC serotype O157:H7.

EspA filaments have been shown to promote attachment of EPEC and EHEC to cultured cells (Ebel et al 1998; Knutton et al 1998). This data also support an adhesive role for EspA filaments since bacterial-RBC attachment appears to depend solely on the presence of EspA filaments. Strains which lacked other recognised EPEC adhesins (Bfp, intimin) but which produced EspA filaments adhered to RBCs as effectively as wildtype EPEC whereas strains which were unable to produce EspA filaments (espa, espd mutants) did not adhere to human RBCS. This contrasts with adhesion to cultured cells where espA and espd mutants do adhere in localised aggregates, probably mediated by Bfp. If this is the case, this would indicate that Bfp receptors are lacking on RBC membranes. Based on osmoprotection experiments, Shigella was recently shown to insert a ˜22 Å hydrophilic pore in the RBC membrane (Blocker et al 1999); the osmoprotection experiments reported here are consistent with a similar mechanism for EPEC and EHEC-induced haemolysis although the detailed analysis to determine a predicted pore size has yet to be performed. Five EPEC proteins, EspA, EspB, EspD, EspF and Tir, have been shown to be secreted by the EPEC type III secretion system and EspB, EspD and Tir have. been shown to be translocated to the host cell membrane (Kenny et al 1997; Wachter et al 1999; Wolff et al 1998). Based on homology with the pore-forming proteins secreted by Yersinia and Shigella respectively, EspB and EspD have been proposed as candidate pore-forming proteins secreted by EPEC (Frankel et al 1998). In this study EspD was required for EPEC-induced haemolysis and was the only RBC membrane-associated protein detected following haemolysis suggesting that EspD may play an important role in pore-formation. EspB, on the other hand, was detected very faintly or not at all in the RBC membrane fraction following haemolysis. In the case of Yersinia and Shigella, both YopB/YopD and IpaB/IpaC are required for pore formation (Blocker et al 1999; Neyt and Cornelis, 1999). It could also be the case with EPEC that both EspB and EspD are involved in pore formation but that EspB was not consistently detected in this study due to the sensitivity of our assay system. However, the observation that an espB mutant still produced normal EspA filaments, adhered to RBCs by EspA filaments and caused some haemolysis suggests that EspB is required for full haemolytic activity but is not an absolute requirement for haemolysis. Contrary to our observations, Warawa et al. (Warawa et al 1999), using a contact-dependent haemolysis assay, reported that an espB mutant was non-haemolytic. This difference possibly reflects our more sensitive haemolysis assay which yielded >90% haemolysis with wildtype strains compared to a maximum of only 15% in the Warawa study. In conclusion, this study has shown that intimate bacterial-RBC membrane contact is not a requirement for EPEC-induced haemolysis. The data also provides further support for a model of the EPEC translocon in which pores in the bacterial and host cell membrane are connected by hollow EspA filaments and suggests that EspD may be the major component of a translocation pore inserted in the host cell membrane.

Experimental Procedures

Bacterial strains and plasmids. The EPEC and EHEC strains and plasmids used in this study are listed in Table 1. Stock cultures of the strains were subcultured in Luria broth or Luria broth supplemented with kanamycin (100 μl/ml) or ampicillin (100 μl/ml) as appropriate and incubated aerobically for 18 h at 37° C.

TABLE 1
List of strains and plasmids

| | Description | Reference |
|---|---|---|
| Strain | | |
| E2348/69 | Wildtype EPEC | (Levine et al 1985) |
| JPNI5 | EAF-plasmid cured | (Jerse et al 1990) |
| CVD206 | eaeA- | (Donnenberg & Kaper, 1991) |
| UMD864 | espB- | (Donnenberg et al 1993) |
| UMD872 | espA- | (Kenny et al 1996) |
| UMD870 | espD- | (Lai et al 1997) |
| UMD874 | espF- | (McNamara & Donnenberg, |
| | | 1998) |
| CVD465 | escC- | L A Wainwright & J B Kaper |
| ΔTir | tir- | (Kenny et al 1997) |
| 85-170 | EHEC(O157:H7) | (Tzipori et al 1987) |
| Plasmid | | |
| pMSD2 | cloned espA | (Kenny, et al 1996) |
| pMSD3 | cloned espB | (Donnenberg, et al 1993) |
| PLCL 123 | cloned espD | (Lai, et al 1997) |
| pLAW215 | cloned escC | L A Wainwright & J B Kaper |
| pICC25 | pMSD2 harbouring an | (Delahay et al 1999) |
| | Arg¹⁶³ mutation | |
| pICC27 | pMSD2 harbouring | (Delahay et al 1999) |
| | Arg¹⁴⁹/Arg¹⁶³ mutations | |

Haemolysis Assay
Assay Protocol

Human blood was obtained from laboratory volunteers. Red blood cells (RBC) were sedimented, washed 3× in phosphate buffered saline (PBS) and a 3% suspension added to polylysine coated 30mm tissue culture dishes for 20 min. Non-attached RBCs were removed by further washing with PBS and the resulting RBC monolayer covered with 2 ml of HEPES buffered Dulbecco's Modified Eagle's Medium (DMEM) without phenol red. Thirty microlitres of an overnight broth culture was added to each RBC monolayer and the dishes incubated for up to 6 hours at 37° C. after which the culture medium was transferred to a microfuge tube and bacteria sedimented. Supernatants were monitored for haemoglobin release by measuring the optical density at 543 nm. Supernatants fronm uninfected RBC monolayers incubated under the same conditions were used to provide a baseline level of haemolysis (B); total haemolysis (T) was obtained from monolayers incubated with a 30-fold dilution of PBS. Percentage haemolysis (P) was calculated from: P=[(X-B)/(T-B)]×100 where X is the optical density of the sample analysed. Contact-dependent haemolysis was performed essentially as above except dishes were incubated for 2 h, centrifuged (2,000 g×5 min) then incubated for a further 2 h. The results are the mean of three independent experiments. Errors given are standard deviations.

Osmoprotection Studies

Haemolysis assays were performed as described above except that 30 mM osmoprotectant (sucrose (M.Wt. 350); raffinose (M.Wt. 600); PEG (M.Wt. 1000, 2000, and 3000)) was added to the assay medium.

RBC Membrane Isolation

The method for membrane isolation was adapted from Blocker et al. (Blocker et al 1999). Human blood was obtained from laboratory volunteers. Tris buffered saline (TBS) containing a protease inhibitor cocktail (Roche) was used throughout the procedure. RBCs were pelleted, and washed 3× with TBS. The sedimented RBCs were resuspended in TBS at approximately 5×10⁸ cells/ml. Two ml of washed RBCs were then mixed with 0.4 ml of a 37° C. overnight stationary culture of each of E2348/69, UMD872, UMD864 and UMD870. 2.4 ml of TBS was added and the infections incubated at 37° C. for 6 h. 0.8 ml of distilled water was then added to obtain equal lysis in each infection. The samples were vortexed, and spun to pellet any unlysed RBCS. 3.4 ml of supernatant was then mixed with 6 ml of 72% sucrose and transferred to SW40 centrifuge tubes. The mixtures were then overlaid with 2 ml 42% sucrose and 1 ml of 25% sucrose. The sucrose cushions were then spun at 15,000g for 16 h. The RBC membranes were collected from the 25/42% sucrose interface and spun at 45,000 g for 30 min. at 4° C. The RBC membrane pellets were finally resuspended in 40 μl of TBS.

Immunoblotting

10 μl of the RBC membrane preparation from each of the E2348/69, UMD872, UMD864 and UMD870 infections were separated by SDS-PAGE, and transferred to nitrocellulose membrane. The membrane was blocked with 10% skimmed milk in PBS, 0.05% Tween-20 for 1 h. The membranes were then washed in PBS-Tween, incubated with either anti-EspA, anti-EspB, anti-EspD or anti-Tir monoclonal antibodies diluted 1:100 in PBS-Tween, overnight at 4° C. (Hartland et al 2000). The blots were washed and detected with antimouse-AP conjugated antibody as previously described (Knutton et al 1998).

Microscopy

Light Microscopy

RBC monolayers were examined for lysis and bacterial adhesion by phase contrast microscopy. following the removal of non-adherent bacteria by washing with PBS. Quantitation of adhesion was performed using phase contrast micrographs and counting numbers of bacteria adhering to 100 RBCS. The results, expressed as numbers of bacteria/RBC, are the mean of 2 separate experiments; errors given are standard deviations.

Immunofluorescence

Immunofluorescence was performed as previously described on fixed and permeabilized RBCs using polyclonal EspA and monoclonal EspB and. EspD antisera (Knutton et al 1998; Wolff et al 1998).

Electron Microscopy

For immunogold labelling of cell-associated bacteria, RBC monolayers on plastic (Therminox) cover slips were briefly fixed for 10 min. in 0.1% glutaraldehyde, washed, and incubated with EspA or EspD antiserum (1:100) for 4 hours at room temperature. Cells were washed and incubated with 10-nm gold-labelled goat anti-mouse serum for 12 hours at 4° C. After further thorough washing, cells were fixed in 3% buffered glutaraldehyde and processed for thin-section electron microscopy using standard procedures (Knutton, 1995). Samples were examined in a Jeol 1200EX electron microscope operated at 80 kV.

For scanning electron microscopy RBC monolayers prepared on glass cover slips were fixed with 3% glutaraldehyde, post-fixed in 1% osmium tetroxide, dehydrated through graded acetone solutions and critical point dried. For immunogold labelling, monolayers were briefly fixed for 10 min. in 0.1% glutaraldehyde, washed, incubated with EspA antiserum (1:100) for 2 hour at room temperature, washed again and incubated with 30-nm gold-labelled goat anti-rabbit serum for 2 hours. After further thorough washing, cells were fixed in 3% buffered glutaraldehyde and processed as described above. Mounted specimens were sputter coated with platinum (Polaron Ltd) and examined in a Jeol 1200EX Scanning Transmission EM.

References

Blocker, A., Gounon, P., Larquet, E., Niebuhr, K., Cabiaux, V., Parsot, C. & Sansonetti, P. (1999). The tripartite type III secreton of Shigella flexneri inserts IpaB and IpaC into host membranes. Journal of Cell Biology 147, 683-93.
Clerc, P., Baudry, B. & Sansonetti, P. J. (1986). Plasmid-mediated contact haemolytic activity in Shigella species: correlation with penetration into HeLa cells. Annales de L Institut Pasteur—Microbiologie 137A, 267-78.
Deibel, C., Kramer, S., Chakraborty, T. & Ebel, F. (1998). EspE, a novel secreted protein of attaching and effacing bacteria, is directly translocated into infected host cells, where it appears as a tyrosine-phosphorylated 90 kDa protein. Molecular Microbiology 28, 463-74.
Delahay, R. M., Knufton, S., Shaw, R. K., Hartland, E. L., Pallen, M. J. & Frankel, G. (1999). The coiled-coil domain of EspA is essential for the assembly of the type III secretion translocon on the surface of enteropathogenic Escherichia coli. Journal of Biological Chemistry 274, 35969-74.
Donnenberg, M. S. & Kaper, J. B. (1991). Construction of an eae deletion mutant of enteropathogenic Escherichia coli by using a positive-selection suicide vector. Infection & Immunity 59, 4310-7.
Donnenberg, M. S., Yu, J. & Kaper, J. B. (1993). A second chromosomal gene necessary for intimate attachment of enteropathogenic Escherichia coli to epithelial cells. Journal of Bacteriology 175, 4670-80.
Ebel, F., Podzadel, T., Rohde, M., Kresse, A. U., Kramer, S., Deibel, C., Guzman, C. A. & Chakraborty, T. (1998). Initial binding of Shiga toxin-producing Escherichia coli to host cells and subsequent induction of actin rearrangements depend on filamentous EspA-containing surface appendages. Molecular Microbiology 30, 147-61.
Frankel, G., Phillips, A. D., Rosenshine, I., Dougan, G., Kaper, J. B. & Knutton, S. (1998). Enteropathogenic and enterohaemorrhagic Escherichia coli: more subversive elements. Molecular Microbiology 30, 911-21.
Giron, J. A., Ho, A. S. & Schoolnik, G. K. (1991). An inducible bundle-forming pilus of enteropathogenic Escherichia coli. Science 254, 710-3.
Hakansson, S., Schesser, K., Persson, C., Galyov, E. E., Rosqvist, R., Homble, F. & WolfWatz, H. (1996). The YopB protein of Yersinia pseudotuberculosis is 5 essential for the translocation of Yop effector proteins across the target cell plasma membrane and displays a contact-dependent membrane disrupting activity. EMBO J 15, 5812-23.
Hartland, E. L., Daniell, S. J., Delahay, R. M., Neves, B. C., Wallis, T., Shaw, R.

K., Hale, C., Knutton, S. & Frankel, G. (2000). The type III protein translocation system of enteropathogenic Escherichia coli involves EspA-EspB protein interactions. Molecular Microbiology 35, 1483-1492.
Hueck, C. J. (1998). Type III protein secretion systems in bacterial pathogens of animals and plants. Microbiology & Molecular Biology Review (Washington, D.C.) 62, 379-433.
Jarvis, K. G., Giron, J. A., Jerse, A. E., McDaniel, T. K., Donnenberg, M. S. & Kaper, J. B. (1995). Enteropathogenic Escherichia coli contains a putative type III secretion system necessary for the export of proteins involved in attaching and effacing lesion formation. Proc Natl Acad Sci USA 92,7996-8000.
Jerse, A. E., Martin, W. C., Galen, J. E. & Kaper, J. B. (1990). Oligonucleotide probe for detection of the enteropathogenic Escherichia coli (EPEC) adherence factor of localized adherent EPEC. Journal of Clinical Microbiology 28, 2842-4.
Jerse, A. E., Yu, J., Tall, B. D. & Kaper, J. B. (1990). A genetic locus of enteropathogenic Escherichia coli necessary for the production of attaching and effacing lesions on tissue culture cells. Proc Natl Acad Sci USA 87, 7839-43.
Kenny, B., DeVinney, R., Stein, M., Reinscheid, D. J., Frey, E. A. & Finlay, B. B. (1997). Enteropathogenic E. coli (EPEC) transfers its receptor for intimate adherence into mammalian cells. Cell 91, 511-20.
Kenny, B., Lai, L. C., Finlay, B. B. & Donnenberg, M. S. (1996). EspA, a protein secreted by enteropathogenic Escherichia coli, is required to induce signals in epithelial cells. Molecular Microbiology 20, 313-23.
Knutton, S. (1995). Electron microscopical methods in adhesion. Methods in Enzymology 253, 145-58.
Knutton, S., Baldini, M. M., Kaper, J. B. & McNeish, A. S. (1987). Role of plasmid-encoded adherence factors in adhesion of enteropathogenic Escherichia coli to HEp-2 cells. It?fection & Immunity 55, 78-85.
Knutton, S., Rosenshine, I., Pallen, M. J., Nisan, I., Neves, B. C., Bain, C., Wolff, C., Dougan, G. & Frankel, G. (1998). A novel EspA-associated surface organelle of enteropathogenic Escherichia coli involved in protein translocation into epithelial cells. EMBO J 17, 2166-2176.
Kubori, T., Matsushima, Y., Nakamura, D., Uralil, J., Lara-Tejero, M., Sukhan, A., Galan, J. E. & Aizawa, S. 1. (1998). Supramolecular structure of the Salmonella typhimurium type III protein secretion system. Science 280, 602-5.
Lai, L. C., Wainwright, L. A., Stone, K. D. & Donnenberg, M. S. (1997). A third secreted protein that is encoded by the enteropathogenic Escherichia coli pathogenicity island is required for transduction of signals and for attaching and effacing activities in host cells. Infect Immun 65, 2211-7.
Levine, M. M., Nataro, J. P., Karch, H., Baldini, M. M., Kaper, J. B., Black, R. E., Clements, M. L. & O'Brien, A. D. (1985). The diarrheal response of humans to some classic serotypes of enteropathogenic Escherichia coli is dependent on a plasmid encoding an enteroadhesiveness factor. J Infect Dis 152, 550-9.
McDaniel, T. K. & Kaper, J. B. (1997). A cloned pathogenicity island from enteropathogenic Escherichia coli confers the attaching and effacing phenotype on E. coli K-12. Mol Microbiol 23, 399-407.
McNamara, B. P. & Donnenberg, M. S. (1998). A novel proline-rich protein, EspF, is secreted from enteropathogenic Escherichia coli via the type III export pathway. FEMS Microbiology Letters 166, 71 -8.
Neyt, C. & Cornelis, G. R. (1999). Insertion of a Yop translocation pore into the macrophage plasma membrane by Yersinia enterocolitica: requirement for translocators YopB and YopD, but not LcrG. Molecular Microbiology 33, 971-8 1.
Tamano, K., Aizawa, S. I., Katayama, E., Nonaka, T., Imajoh-Ohmi, S., Kuwae, A., Nagai, S. & Sasakawa, C. (2000). Supramolecular structure of the Shigella type III secretion machinery: the needle part is changeable in length and essential for delivery of effectors. EMBO J 19, 3876-3887.
Tzipori, S., Karch, H., Wachsmuth, K. I., Robins-Browne, ]Z. M., O'Brien, A. D., Lior, H., Cohen, M. L., Smithers, J. and Levine, M. M. (1987). Role of a 60-megadalton plasmid and Shiga-like toxins in the pathogenesis of infection caused by enterohemorrhagic Eschericvhia coli 0157:H7 in gnotobiotic piglets. Infection & Immunity 55, 3117-3125.
Wachter, C., Beinke, C., Mattes, M. & Schmidt, M.. A. (1999). Insertion of EspD into epithelial target cell membranes by infecting enteropathogenic Escherichia coli. Molecular Microbiology 31, 1695-707.
Warawa, J., Finlay, B. B. & Kenny, B. (1999). Type III secretion-dependent hemolytic activity of enteropathogenic Escherichia coli. Infection & Immunity 67, 5538-40.
Wolff, C., Nisan, I., Hanski, E., Frankel, G. & Rosenshine, 1. (1998). Protein translocation into HeLa cells by infecting enteropathogenic Escherichia coli. Mol. Microbiol 28, 143-155.

### EXAMPLE 2

### Using Microarrays in the Study of Alterations in Bacterial Gene Expression during Cell Attachment

DNA microarrays are used to study alterations in bacterial gene expression during Type III secretion system (TTSS)-mediated cell attachment and A/E lesion formation. DNA microarrays offer the potential to monitor global changes in gene expression at the genome level. Single stranded DNA molecules, in the form of PCR product or oligonucleotides are immobilised onto a treated glass matrix. Bacterial RNA is then isolated from a control and an experimental system, and fluorescently labelled cDNA is generated by an indirect method that gives superior Cy dye incorporation; amino-allyl dUTP is incorporated by use of random hexamers as primers. Cy3 and Cy5 monoesters are then bound to the cDNA by a simple chemical reaction (described, for example, at http:cmgm.stanford.edu/pbrown/protocols). The two labelled cDNA samples are then mixed and hybridised to the array and the amount of cDNA bound to each spot is quantified using a microarray scanner. Glass microarrays may offer the advantage of accurate comparative measurements due to the fact that both the control and test cDNAs may be hybridised to a single array. Measurement of the relative fluorescent signals indicates whether genes are up-regulated, down-regulated, constitutively expressed or not expressed.

E. coli DNA microarrays are manufactured and cDNA hybridisations and scanning performed. DNA microarrays allow the monitoring of global changes in gene expression at the genome level. E. coli. DNA microarrays are manufactured, for example using the same approach pioneered by Pat Brown and Joe De Risi at Stanford, USA. Gene-specific PCR products for E. coli K12 and for specific E. coli virulence genes are produced with an MWG RoboAmp 4200 liquid handling robot, using commercial primer pairs (E. coli K12 primers have already been used successfully for microarray construction by Richmond et al. (Richmond, C. S., J. D. Glasner, R. Mau, H. Jin, and F. R. Blattner. (1999) Genome-wide expression profiling in Escherichia coli K-12. Nucleic Acids Res. 27:3812-3835) and were purchased from Sigma-Genosys; E. coli virulence gene primers may be designed, for example using ArrayMaker software). PCR products are gridded as 170 μm spots using split pins (Majer Precision) on a Stanford microarraying robot (http://cmgm.stanford.edu/pbrown/mguide/index.html) and immobilised onto a treated glass matrix (Corning GAPSTM). The microarrays may contain the entire E. coli K12 genome, plus EPEC and EHEC virulence genes including the LEE locus, espC/P, hlyA, lif, perA, bfpA and other EPEC and EHEC plasmid genes, as well as the complete Shigella flexneri virulence plasmid (which was recently sequenced at the Pasteur Institute). As information of new E. coli genes (for example EHEC, due shortly) become available these may be added to the array. Following incubation of bacteria with the lipid bilayer, the bacterial mRNA is extracted from control and test samples at varying time points. RNA is initially stabilised within bacterial cells by use of RNAlater (Ambion) or guanidinium isothiocyanate to prevent RNA degradation. Adhering bacteria are isolated by differential wash procedures, and bacterial RNA is prepared by a method that minimises mRNA degradation (Tedin, K., and U. Blasi. (1996) The RNA chain elongation rate of the lambda late mRNA is unaffected by high levels of ppGpp in the absence of amino acid starvation. J Biol Chem. 271:17675-17686).

Bacterial RNA is then isolated, and quantified spectrophotometrically at several wavelengths, from a control and an experimental system. Fluorescently labelled cDNA is generated by an indirect method that gives superior Cy dye incorporation; amino-allyl dUTP is incorporated by use of random hexamers as primers. Cy3 and Cy5 monoesters are then bound to the cDNA by a simple chemical reaction (http://cmgm.stanford.edu/pbrown/protocols/). The two labelled cDNA samples are. then mixed and hybridised to the array and the amount of cDNA bound to each spot is quantified using a microarray scanner. Glass microarrays may be used which offer the advantage of accurate comparative measurements due to the fact that both the control and test cDNAs are hybridised to a single array. Measurement of the relative fluorescent signals indicates whether genes are up-regulated, down-regulated, constitutively expressed or not expressed. Data analysis will be performed with GeneSpring data mining software.

Changes in gene expression between EPEC and EHEC and between wildtype and defined mutants during the early stages of infection are compared using the lipid bilayer infection model. The use of lipid bilayers, for example in the form of RBCs or RBC ghosts or vesicles has a tremendous advantage as it avoids the difficulties of having relatively high quantity of mammalian RNA in the preparation. If RBC ghosts or vesicles or other membrane preparations, or artificial lipid bilayers are used rather than RBCs, the amount of mammalian protein that is present is also reduced. This may help in relation to sample preparation/handling and/or in analysing changes in protein levels.

The RBC/lipid bilayer system allows monitoring of changes in gene expression during early stages of the infection when the TTSS is active but the host cell is not responding to the infection (no A/E lesions). Binding of EPEC and EHEC to the surface of the RBCs/lipid bilayer is mediated solely by EspA filaments and this is followed by protein translocation. Accordingly, this assay selects for a synchronised adherent bacterial population, all of which are at the same “developmental stage” where the TTSS is active. This may be of particular benefit with relation to EPEC and EHEC because the direct association of TTSS with plasma membrane can be seen and studied. The effect of defined mutations that modulate the outcome of the lipid bilayer/RBC-EPEC interaction are determined and compared between themselves and the parent strain. This allows determination of whether changes in LEE gene expression occurs in EPEC following TTSS-mediated cell attachment (inoculum of espA- or espB- strains compared with adherent espB- strain) and protein translocation (adherent espB-strain compared with adherent wildtype strain). The effect of per (and other global regulators) and absence of Tir or surface intimin on bacterial gene expression while the TTSS is involved in protein translocation may also be studied in the RBC. As this system provides a model to study real time changes in gene expression within a bacterium employing a TTSS to translocate proteins, the results may be useful in relation to infections other than EPEC or EHEC infection.

In contrast to interaction with RBCs, infection of HEp-2 or Caco-2 cells with wild type EPEC and EHEC leads to intimin-Tir-mediated intimate attachment and A/E lesion formation. Using a similar approach to that described above for the lipid bilayer/RBC, we will use DNA microarrays to determine how these subsequent events affect bacterial gene expression although in this model optimisation due to the presence of HEp-2 RNA in the RNA preparations may be required. This may indicate the changes in gene expression taking place during different stages of the infection. We hypothesise, from the fact that the host cell is responding to the infection, that this will have an effect on the dynamic crosstalk with the bacteria (we have already shown that EPEC responds to A/E lesion formation by down regulating EspA filaments (47) and intimin (79)).

It is likely that the LEE-gene products-mediated interaction with eukaryotic cells affects expression of non-LEE genes (metabolic, iron acquisition etc). We have observed that although bacteria in the inoculum are highly motile, we could not detect any falgellated bacteria associated with RBC or HEp-2 cells. This suggests that expression of TTSS and flagellar genes are coordinately regulated. This is investigated in more detail using the whole-genome E. coli K12 microarray, which carries the additional virulence genes of interest.

In summary, the high conservation of the majority of EPEC and EHEC genes allows the use of heterologous hybridisation with E. coli K12 genes to visualise expression. A specific EPEC or EHEC array may also be used. Following investigation of global regulation of gene expression at different stages of the interaction of EPEC and EHEC with the host cell, the role of selected gene products in entero-pathogenesis may be studied further using known methodologies and a range of infection models. These gene products may be, or may allow the identification of, new candidates for vaccine development and anti microbial targets.

1. A method of analysing gene expression occurring in a microorganism before, during or after contact with or adhesion of the microorganism to a lipid bilayer, comprising the step of exposing the microorganism to a lipid bilayer, wherein the lipid bilayer is substantially not associated with protein or RNA synthetic machinery.

2. The method of claim 1 wherein the microorganism invades through the lipid bilayer.

3. A method of claim 1 or 2 wherein the lipid bilayer is comprised in a red blood cell or membrane derived therefrom.

4. A method of claim 1 or 2 wherein the lipid bilayer comprises an artificial lipid bilayer.

5. A method of claim 1, 2 or 3 wherein the lipid bilayer comprises a purified cell membrane.

6. A method of claim 3 wherein the membrane derived from a red blood cell is a red blood cell ghost.

7. A method of claim 3 or 6 wherein the red blood cell is a human red blood cell.

8. A method of claim 4 wherein the composition of the lipid bilayer resembles that of a red blood cell.

9. A method of any of the preceding claims comprising the step of separating any microorganisms bound to the lipid bilayer from any microorganisms not so bound, following exposure of the microorganisms to the lipid bilayer.

10. A method of claim 9 comprising the step of comparing gene expression in the said microorganisms bound to the lipid bilayer with gene expression in the said microorganisms not so bound.

11. A method of any one of the preceding claims wherein gene expression is analysed using a nucleic acid microarray.

12. A method of any one of the preceding claims wherein gene expression is analysed using a protein microarray.

13. A method according to any one of the preceding claims further comprising the step of determining whether any microorganism component has been transferred to the lipid bilayer.

14. A method of analysing the interaction between a microorganism and a lipid bilayer, comprising the steps of exposing the microorganism to a lipid bilayer, wherein the lipid bilayer is substantially not associated with protein or RNA synthetic machinery, and determining whether any microorganism component has been transferred to the lipid bilayer.

15. The method of claim 14 wherein the lipid bilayer is as defined in any one of claims 3 to 8.

16. A method of any one of the preceding claims wherein the microorganism is a bacterium.

17. A method according to claim 16 wherein the bacterium is pathogenic to animals.

18. A method according to any one of claims 1 to 15 wherein the microorganism is a fungus pathogenic to animals.

19. A method according to claim 16 or 17 wherein the bacterium is an E. coli bacterium.

20. A method according to claim 19 wherein the E. coli bacterium is an enterohaemorrhagic E. coli (EHEC) or an enteropathogenic E. coli (EPEC).

21. A method according to claim 20 wherein the bacterium is EPEC strain E2348/69 or EHEC strain 85-170 (0157:H7).

22. A method according to claim 16 or 17 wherein the bacterium is a Helicobacter pylori bacterium.

23. A method according to claim 16 or 17 wherein the bacterium is any one of Bordetella pertussis, Campylobacter jejuni, Clostridium botulinum, Haemophilus ducreyi, Haemophilus influenzae, Klebsiella pneumoniae, Legionella pneumophila, Listeria spp., Neisseria gonorrhoeae, Neisseria meningitidis, Pseudomonas spp., Salmonella spp., Shigella spp., Staphylococcus aureus, Streptococcus pyogenes, Streptococcus pneumoniae, Vibrio spp., and Yersinia pestis.

24. A method according to claim 18 wherein the fungus is any one of Aspergillus spp., Cryptococcus neoformans and Histoplasma capsulatum.

25. A method according to any of the preceding claims wherein the method is performed on a first microorganism and on a second microorganism, wherein the second microorganism differs from the first microorganism substantially only in relation to a component involved or considered to be involved in adhesion of the microorganism to a lipid bilayer.

26. The method of claim 25 wherein the first microorganism is an EPEC wild-type in relation to EspA and the second microorganism is an EPEC that differs from the first microorganism substantially only in relation to EspA.

27. The method of claim 25 wherein the first microorganism is an EPEC wild-type for EspB and the second microorganism is an EPEC that differs from the first test organism substantially only in relation to EspB.

28. A kit of parts comprising a lipid bilayer substantially not associated with protein or RNA synthetic machinery, and a nucleic acid microarray and/or protein microarray.

29. A method of identifying a gene of a microorganism, the expression of which differs in the presence or absence of contact and/or adhesion of the microorganism to a lipid bilayer, the method comprising performing the method of any one the preceding claims, and further comprising the step of comparing the expression of at least one gene in the presence and absence of said contact and/or adhesion, and selecting a. gene whose expression is different in the presence and absence of after contact and/or adhesion of the microorganism to a lipid bilayer.

30. A method of selecting a target for development or identification of an antiinfective agent or vaccine, wherein a method according to claim 29 is performed and a product of a gene whose expression is identified as differing in the presence and absence of contact and/or adhesion is selected as a target.

31. A microorganism in which a gene identified using a method according to claim 29 is mutated or overexpressed.

32. A gene identified using a method according to claim 29.

33. A polypeptide encoded by a gene according to claim 32.

34. A method of identifying a compound which reduces the ability of a microorganism to adhere to a host cell comprising the step of selecting a compound which interferes with the function of a gene according to claim 32 or a polypeptide according to claim 33.

35. A compound identified or identifiable by the method of claim 34.

36. Use of a compound according to claim 35 for treating infection of a host organism with said microorganism.

37. A molecule which selectively interacts with, and substantially inhibits the function of, a gene according to claim 32 or nucleic acid product thereof, or a polypeptide according to claim 33.

38. A molecule according to claim 37 which is an antisense nucleic acid or nucleic acid derivative or an antibody.

39. A molecule according to claim 37 or 38 which is an antisense oligonucleotide.

40. A molecule according to any one of claims 37 to 39 or compound according to claim 35 or polypeptide according to claim 33 or polynucleotide encoding said polypeptide for use in medicine.

41. A method of treating a host which has, or is susceptible to, an infection with a microorganism, the method comprising administering an effective amount of a molecule, compound, polypeptide or polynucleotide as defined in claim 40 wherein said gene is present in said microorganism, or a close relative of said microorganism.

42. The use of a polypeptide according to claim 33 or polynucleotide encoding said polypeptide or microorganism overexpressing same in the manufacture of a medicament for vaccination of a host which has or is susceptible to, an infection 70 with a microorganism, wherein said gene is present in said microorganism, or a close relative of said microorganism.

43. A pharmaceutical composition comprising a molecule, compound, polynucleotide or polypeptide as defined in claim 40, or a microorganism as defined in claim 42, and a pharmaceutically acceptable carrier.

44. A method of microorganism, for example bacterial, detection and/or characterisation wherein the presence/absence and/or expression of a gene identifiable by the method of claim 29 in a sample is determined.

45. The method of claim 44 wherein the presence of expression of a said gene is determined by determination of the presence of a phenotypic characteristic in the sample.
